(19) United States
(12) Patent Application Publication
(10) Pub. No.: US 2023/0098307 A1
(43) Pub. Date: Mar. 30, 2023

(54) VITAMIN B6-COUPLED POLYOL-BASED POLYDIXYLITOL GENE TRANSPORTER COMPRISING PEPTIDE BINDING SPECIFICALLY TO CANCER STEM CELL AND CANCER STEM CELL-TARGETED THERAPY TECHNIQUE

(71) Applicant: ELBIO INC, Seoul (KR)

(72) Inventors: Pankaj GARG, Seoul (KR); Shambhavi PANDEY, Seoul (KR); Myung Chul LEE, Hanam-si (KR); Bog Hee KIM, Seoul (KR)

(21) Appl. No.: 17/802,509

(22) PCT Filed: Mar. 13, 2020
(86) PCT No.: PCT/KR2020/003566

§ 371 Date: Aug. 25, 2022

(30) Foreign Application Priority Data
Mar. 13, 2020 (KR) 10-2020-0031434

(51) Int. Cl. A61K 48/00; C12N 15/113; C12N 15/90; C12N 15/11; C12N 9/22; A61P 35/00

(52) U.S. Cl. CPC A61K 48/0041; C12N 15/1135; C12N 15/907; C12N 15/11; C12N 9/22; A61P 35/00; C12N 2310/20; C12N 2800/80; C12N 2310/14

(57) ABSTRACT

Provided is a polydixylitol polymer gene transporter (VBXYP-P) containing vitamin B6 and a cancer stem cell-targeting peptide (TR-7) and a method for preparing the same. In addition, provided is a nucleic acid delivery complex in which a therapeutic nucleic acid is conjugated to the gene transporter, and a pharmaceutical composition for gene therapy containing the complex as an active ingredient. In addition, provided is the gene transporter, a gene delivery complex, and gene therapy using the same. It was observed that VBXYP-P of the present disclosure had a remarkably higher nucleic acid delivery rate to cancer stem cells than the pre-existing nucleic acid transporter, and when VBXYP-P was conjugated with DNA, the complex was almost free of cytotoxicity and permeated a blood brain barrier to exhibit remarkably high transformation efficiency for cancer stem cells inside a brain tumor.

### TECHNICAL FIELD

The present disclosure relates to a method for preparing a complex (VBXYP-P) in which a cancer stem cell-targeting peptide (TR-7 peptide) is coupled to a vitamin B6-coupled polydixylitol polymer gene transporter (VB-PdXYP or VBXYP). In addition, the present disclosure relates to a nucleic acid delivery complex in which a therapeutic nucleic acid is electrostatically bound to the gene transporter, and a pharmaceutical composition for gene therapy containing the complex as an active ingredient. In addition, the present disclosure relates to the gene transporter, a gene delivery complex, and cancer stem cell therapy using the same.

### BACKGROUND ART

Glioblastoma multiforme that is grade 4 astrocytoma is the most common and malignant brain tumor. In the case of the glioblastoma multiforme, the prognosis is poor and the survival rate is low. Since invasive tumors cannot be completely eliminated by only surgical treatment, chemoradiotherapy is primarily attempted to prevent recurrence. However, some cancer stem cells at a stage of latency may avoid the treatment and then proliferate and metastasize to generate new tumors resistant to chemoradiotherapy, which is the cause of tumor recurrence. In order to prevent the tumor recurrence and completely treat the glioblastoma multiforme, there is a need to target and eliminate cancer stem cells from the inside of the tumor. However, cancer stem cells overexpress a drug efflux transporter to develop resistance to a chemotherapeutic drug, thereby nullifying the therapeutic drug. In order to overcome these difficulties, research on genome editing therapy for cancer stem cells using a clustered regularly interspaced short palindromic repeats and CRISPR-associated protein 9 (CRISPR-Cas9) technology, and a small interfering RNA (siRNA) technology is attracting attention as an alternative cancer therapy. Recently, the CRISPR-Cas9 gene editing technology allows addition, deletion, or alternation of a genetic material at a specific location in genome, and is faster, cheaper, more accurate, and more efficient than other pre-existing genome editing methods. A CRISPR-Cas9 gene editing tool includes two main parts. The first part is that single-stranded RNA (sgRNA) guides the gene cutting system to a specific editing site of genome, and the second part is that a Cas9 protein having a DNA catalytic activity cuts the genome of the specific site. At this point, a desired sequence may be inserted or deleted to obtain a desired gene sequence.

An innovative cancer therapy for inducing apoptosis of cancer stem cells by knock-out of a major signaling of the cancer stem cells affecting the survival and recurrence of cancer cells in a tumor using a CRISPR-cas9 system to eliminate a root of the tumor may be suggested. Sonic hedgehog signaling, which is important for the survival and maintenance of cancer stem cells, is mediated by a smoothened membrane protein (smoothened, SMO) that is an intermediate regulator. Therefore, the smoothened is classified as a cancer-causing protein and is also a target for chemotherapy. However, when the cancer stem cells are treated with a chemotherapy drug, the cancer stem cells acquire drug resistance by increasing expression of a drug efflux transporter of the corresponding drug, thereby avoiding conventional anticancer therapy. Therefore, the present inventors suggest a novel method for inducing apoptosis of cancer stem cells by synthesizing a gene transporter capable of targeting the cancer stem cells and inhibiting expression of a fundamental smoothened using a CRISPR-cas9 system. Induction of apoptosis of the entire tumor by inducing the apoptosis of the cancer stem cells through genome gene editing of the cancer stem cells is expected to provide a new alternative for a treatment of malignant tumors. Therefore, a demand for developing a gene transporter designed to deliver nucleic acid therapeutics capable of inducing apoptosis of cancer stem cells to cancer stem cells in glioblastoma multiforme with high efficiency has been continuously required.

However, therapy for brain diseases is usually not effective because therapeutic drugs do not permeate the blood brain barrier (BBB). The blood brain barrier, which is a cerebrovascular structure that limits the delivery of substances from blood to brain tissues, is known to be formed mostly by tight junctions of cerebral capillary endothelium, surround blood vessels, and have impermeability to large molecules such as nucleic acids. Specifically, it is known that fat-soluble substances permeate the blood brain barrier, but non fat-soluble substances such as polar substances and strong electrolytes do not permeate the blood brain barrier well. Although there is an advantage in that the brain tissues are protected from harmful substances by the blood brain barrier, there is a disadvantage in that accessibility to therapeutic substances is lower than that of other tissues in the human body because it blocks the delivery of a radioisotope, a pigment, a drug, or the like, required for the treatment of the brain tissues. Under the circumstances in which even the delivery of polar compounds to brain tissues through the blood brain barrier is not easy, the delivery of nucleic acids that are large molecules having a strong polarity is even more difficult. In addition to the blood brain barrier, biologically-hindering mechanisms, such as degradation by nuclease, immune clearance, difficulty in cell influx, off-target deposition in vivo, make delivery of genes to the brain tissues difficult.

Since genes delivered using most viral vectors are not able to permeate the blood brain barrier by systemic delivery, direct injection/insertion into the brain is generally performed. However, transduction is limited in terms of insertion sites and a direct injection method is problematic in terms of invasiveness into brain tissues. Accordingly, in order to increase the pre-existing delivery efficiency of substances to brain tissues by systemic delivery, there has been an attempt to increase a permeability into the blood brain barrier by an intra-arterial injection of an osmotic agent such as mannitol. Specifically, the tissues were pretreated with hyperosmotic mannitol to loosen the tight junctions between the cells, and then, the cells were treated with various gene/drug delivery vehicles. However, the effect of mannitol was temporary and disappeared after 30 minutes, and the effect disappeared even before the influx of drugs or DNA. In addition, an effect of an overall increase of the permeability into the blood brain barrier was obtained by the systemic delivery of mannitol, and thus, it was not possible to increase the permeability of only particular substances for delivery.

Even when genes have permeated the blood brain barrier, the genes still have to safely go through with cellular uptake and endosomal trapping to be delivered to the target cells. Therefore, in terms of gene therapy for animals, delivery of genes to the target cells of tissues is the biggest obstacle and technical problem to be solved. Therefore, a transporter is required to target cancer stem cells and thus to accurately deliver genes. In order to target cancer stem cells, the present inventors have focused on a specific marker for cancer stem cells such as CD133 that is a transmembrane protein known as prominin-1. Such a transmembrane protein is the most representative of several markers and is expressed on surfaces of cancer stem cells. Several researchers have found antibodies or specific peptides capable of targeting these markers. The present inventors have focused on TR-7 (TISWPPR, SEQ ID NO:1) that is a CD133-specific peptide composed of seven amino acids, and have attempted to synthesize a gene transporter containing the peptide to target CD133 present on the surfaces of cancer stem cells.

A gene transporter should have low or no toxicity and be able to selectively and effectively deliver genes to desired cells. These nucleic acid transporters are largely classified into viral and non-viral nucleic acid transporters. Until recently, for clinical trials, viral vectors having high transduction efficiency have been used as a nucleic acid transporter. However, viral vectors such as retrovirus, adenovirus, and adeno-associated virus not only have complex preparation steps, but also have safety problems such as immunogenicity, infection risks, induction of inflammation, and insertion of non-specific DNA and a problem in that an acceptable DNA size is limited. Therefore, the viral vectors have many limitations to be applied in a human body. Accordingly, at present, non-viral vectors have been spotlighted as a replacement for viral vectors.

Non-viral vectors may be repeatedly administered with a minimal immune response, may be specifically delivered to particular cells, has excellent storage stability, and may be easily mass-produced. Examples of these non-viral vectors include cationic liposomes such as N-[1-(2,3-dioleyloxy)-propyl]-N,N,N-trimethylammonium chloride (DOTMA), alkylammonium, cationic cholesterol derivatives, and gramicidin.

Recently, since cationic polymers among non-viral vectors may form a complex through an ionic bond with anionic DNA, the cationic polymers are receiving a lot of attention. Such cationic polymers include poly-L-lysine (PLL), poly(4-hydroxy-L-proline ester), polyethylenimine (PEI), poly[α-(4-aminobutyl)-L-glycolic acid], polyamidoamine dendrimer, poly[N,N′-(dimethylamino)ethyl]methacrylate (PDMAEMA), and the like, and these polymers condense DNA and form nanoparticles to protect DNA from enzymatic degradation, and allow DNA to penetrate rapidly into cells and to be released from endosomes. Most non-viral vectors have advantages such as biodegradability, low toxicity, non-immunogenicity, and convenience for use, compared to viral vectors, but have problems such as relatively low transfection efficiency and limited particle size.

In particular, most cationic polymers used as non-viral vectors exhibit high transduction efficiency in an in vitro environment having a low serum concentration, but have problems in that transfection efficiency of cationic polymer/gene complexes is significantly reduced by various factors present in serum in an in vivo environment, and thus, genes are not smoothly introduced into cells. This is because excessive positive charges occur on the surfaces of the cationic polymer/gene complexes in vivo to cause non-specific interactions with plasma proteins and blood constituents. Therefore, the transfection efficiency of cationic polymers is significantly reduced in an in vivo environment in which a lot of serum is present, but not in an in vitro environment in which serum-free media or a significantly low concentration of serum is present. When this is applied in vivo as it is, aggregates and accumulations in the lungs, liver, and spleen, and furthermore, opsonization and removal by a reticuloendothelial system may be caused. Thus, the therapeutic application of these cationic polymers may be greatly limited. Polyethylenimine (PEI), which has been most broadly studied as a non-viral vector, also has significantly low in vivo transfection efficiency and has a problem such as high cytotoxicity and low effects on gene expression due to low blood compatibility. Therefore, there is an urgent need for developing a nuclear acid transporter having enhanced transfection efficiency while maintaining the advantages of the pre-existing non-viral vectors.

The present inventors have developed various gene transporters. As a result, the present inventors have found that genes may be efficiently delivered to cancer stem cells and cancer stem cells in glioblastoma multiforme maybe targeted to deliver genes, by coupling vitamin B6 and a peptide (TR-7) binding specifically to cancer stem cells to a polyol-based osmotic gene transporter prepared by combining dixylitol diacrylate with polyethylenimine (PEI), the transporter exhibiting significantly low cytotoxicity and remarkably high transformation efficiency by a high permeability into a blood brain barrier (BBB) due to a xylitol dimer backbone, an increased membrane permeability by an osmotic activity, and a proton sponge effect by stimulated intracellular uptake and a backbone of PEI, thereby completing the present disclosure.

### DISCLOSURE

### Technical Problem

An aspect of the present disclosure may provide, as a gene transporter capable of targeting cancer stem cells, a cancer stem cell-targeting peptide-coupled polydixylitol-based polymer gene transporter having significantly enhanced transfection efficiency without exhibiting cytotoxicity.

Another aspect of the present disclosure may provide a method for preparing the polydixylitol-based polymer gene transporter.

Another aspect of the present disclosure may provide a nucleic acid delivery complex in which a therapeutic nucleic acid is electrostatically bound to the polydixylitol-based polymer gene transporter.

Another aspect of the present disclosure may provide a pharmaceutical composition for gene therapy containing the nucleic acid delivery complex as an active ingredient.

### Technical Solution

As an aspect for achieving the above object, there is provided a gene transporter (VBXYP-P) to which vitamin B6 is coupled using the previously invented polydixylitol polymer (PdXYP) (Chemical Formula 3) as an initial backbone and in which a peptide (TR-7 peptide) binding specifically to a CD133 protein that is a marker for cancer stem cells is contained. The present disclosure is designed to target cancer stem cells and thus to deliver genes by improving polydixylitol polymer gene transporters (PdXYP and VB-PdXYP(VBXYP)) that are the previously developed gene transporters. The gene transporter of the present disclosure may have a structure of the following Chemical Formula 1.

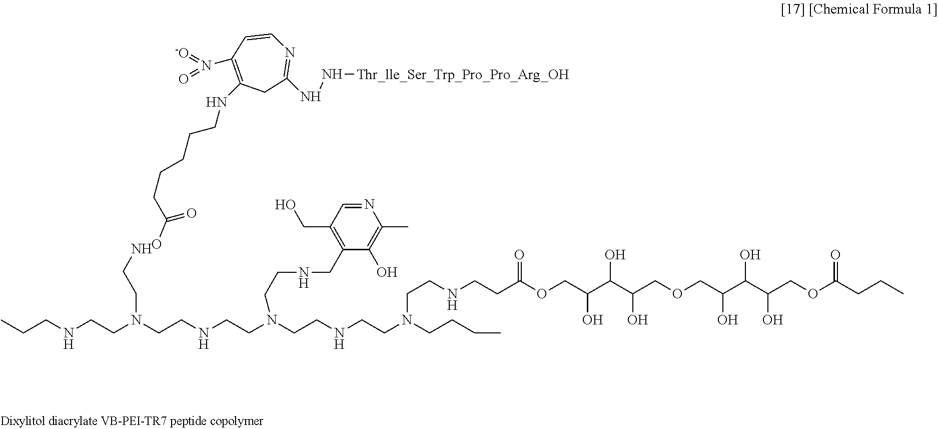

Sulfosuccinimidyl-6-[4′-azido-2′-nitrophenylamino]hex anoate (sulfo-SANPAH) has a structure of Chemical Formula 2. A polydixylitol polymer gene transporter (dixylitol diacrylate VB-PEI-TR7 peptide copolymer, VBXYP-P) in which a cancer stem cell-specific peptide (TR-7 peptide, SEQ ID NO:1) is coupled to the previously developed VB-PdXYP (VBXYP) gene transporter using this linker was prepared.

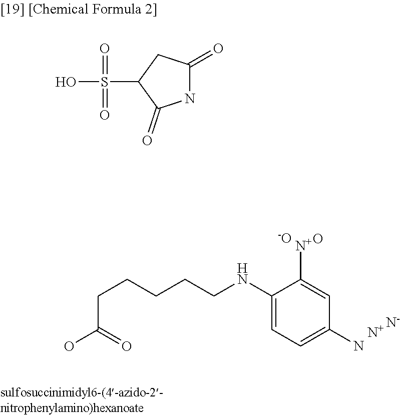

Sulfo-SANPAH of the present disclosure is a heterobifunctional crosslinker. N-Hydroxysuccinimide (NHS) of this linker forms a stable amide bond with a primary amine group of low molecular weight polyethylenimine (PEI) of the transporter in a buffer solution environment with a pH 7 to 9, and nitrophenyl azide is bound to an amine group of TR-7 that is a cancer stem cell-specific peptide through a dehydroazepine intermediate by a reaction of an ultraviolet light of 300 to 460 nm. As a result, VBXYP-P may be prepared (FIG. 3).

The term “TR-7” of the present disclosure refers to a peptide composed of a total of seven amino acids that may specifically bind to a CD133 protein specifically present on a surface of a cancer stem cell. The sequence thereof consists of threonine-isoleucine-serine-tryptophan-proline-proline-argi nine (Thr-Ile-Ser-Trp-Pro-Pro-Arg, SEQ ID NO:1). The term is named “TR-7” after the initials of the first amino acid and last amino acid of this sequence. TR-7 reacts specifically with CD133 present on the surfaces of cancer stem cells, and when the gene transporter containing TR-7 is used, the possibility of targeting the cancer stem cells and delivering a therapeutic nucleic acid increases.

The term “polydixylitol polymer gene transporter (PdXYP)” of the present disclosure refers to the gene transporter patented by the present inventors (10-1809795). Dixylitol is prepared by an acetone/xylitol condensation method, the dixylitol is esterified with acryloyl chloride to prepare dixylitol diacrylate (dXYA), and the dixylitol diacrylate is subjected to a Michael addition reaction with low molecular weight polyethylenimine (PEI), thereby preparing the transporter (FIG. 1).

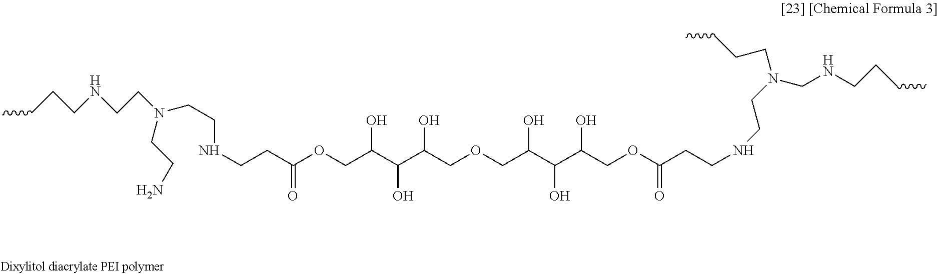

The term “xylitol” refers to a kind of sugar alcohol-based natural sweetener having a chemical formula of C₅H₁₂O₅. The xylitol is extracted from a birch tree, an oak tree, or the like, and has a unique pentose sugar structure. In order to prepare the polydixylitol polymer gene transporter of the present disclosure, dixylitol, a xylitol dimer, is used.

The term “acryloyl chloride” may also be referred to as 2-propenoyl chloride or acrylic acid chloride. The compound reacts with water to form acrylic acid, or reacts with a sodium carboxylate salt to form anhydride, or reacts with an alcohol to form an ester group. In a specific exemplary embodiment in the present disclosure, dixylitol (a xylitol dimer) that is a kind of sugar alcohol is esterified by a reaction with acryloyl chloride to form dixylitol diacrylate (dXYA).

The term “polyethylenimine (PEI)” refers to a cationic polymer having primary, secondary, and tertiary amino groups and a molar mass of 1,000 to 100,000 g/mol, effectively condenses anionic nucleic acids to form colloidal particles, and has high gene deliver efficiency due to its pH responsive buffering ability, such that genes may be effectively delivered to various cells in vitro and in vivo. In the present disclosure, polyethylenimine may be a linear type represented by the following Chemical Formula 4 or a branched type represented by the following Chemical Formula 5, and a molecular weight thereof is a low molecular weight, and preferably 50 to 10,000 Da (based on a weight average molecular weight) in consideration of cytotoxicity. Polyethylenimine is dissolved in water, alcohol, glycol, dimethylformamide, tetrahydrofuran, esters, and the like, and is not dissolved in high molecular weight hydrocarbons, oleic acid, and diethyl ether.

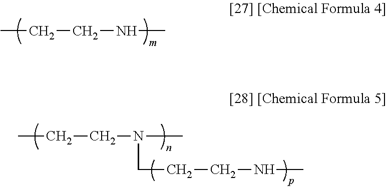

In addition, a final gene transporter backbone of the present disclosure is a polydixylitol polymer gene transporter prepared by additionally coupling vitamin B6 to the polydixylitol polymer gene transporter, so-called a vitamin B6-coupled polydixylitol polymer gene transporter (VB-PdXYP (VBXYP)). The polydixylitol polymer gene transporter to which vitamin B6 is additionally coupled may have a structure of the following Chemical Formula 6.

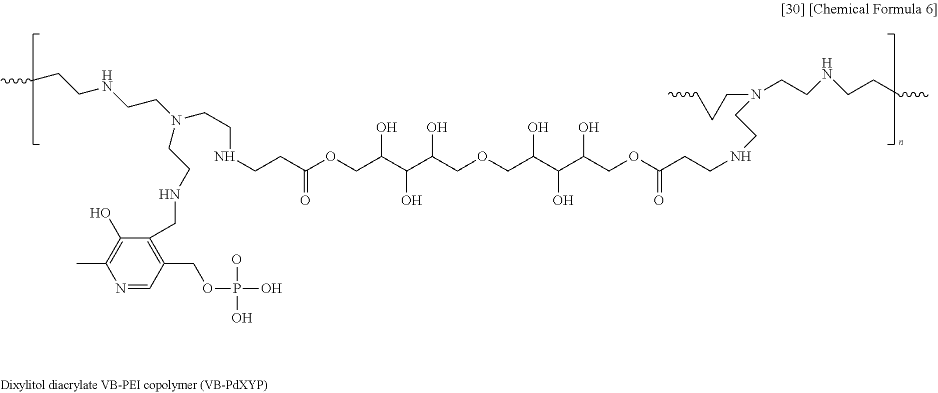

The term “vitamin B6” is present as pyridoxine (PN), pyridoxal (PL), pyridoxamine (PM), or a phosphorylated form of each of PN, PL, and PM (PNP, PLP, or PMP), and is used as a coenzyme for various bioactive enzymes. In particular, when vitamin B6 is used as a coenzyme, vitamin B6 is mainly used in a form of PLP or PMP, and it is known that PLP has a significantly high biological activity. Active vitamin B6 (pyridoxal 5′phosphate (PLP)) of the present disclosure may have a structure of the following Chemical Formula 7.

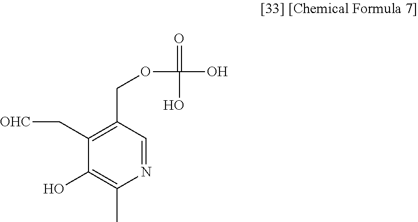

Pyridoxal 5′phosphate (PLP) and the prepared polydixylitol polymer gene transporter are reacted with each other to form a transient Schiff base. Thereafter, the Schiff base is reduced using NaCNBH₄ to obtain a vitamin B6-coupled polydixylitol polymer gene transporter (FIG. 2).

VBXYP-P of the present disclosure induces binding of the transporter to a cell membrane by targeting and binding to CD133 present on the cell membrane of the cancer stem cell, and after the transporter is bound to the cell membrane in this way, introduction of nucleic acids into the cells are efficiently induced by a proton sponge effect by the polydixylitol polymer gene transporter, such that significantly enhanced transformation efficiency may be exhibited. In addition, VBXYP-P has significantly low cytotoxicity, and thus, VBXYP-P maybe efficiently used for gene therapy as a gene transporter. Therefore, VBXYP-P may exhibit high transfection efficiency for cancer stem cells compared to normal cancer cells (FIGS. 8 through 10).

It is preferable that the VBXYP-P gene transporter of the present disclosure has a molecular weight of 1,000 to 100,000 Da (based on a weight average molecular weight) in order to effectively deliver genes. In addition, it is preferable that the nucleic acid delivery complex in which a nucleic acid is electrostatically bound to the gene transporter of the present disclosure has a zeta potential of 1 to 100 mV in order to effectively deliver genes, and in particular, the nucleic acid delivery complex may have a zeta potential of 25 to 50 mV. When physicochemical properties within the above ranges are exhibited, the present gene transporter may be effectively introduced into endosomes of cells.

As another aspect, the present disclosure provides a method for preparing VBXYP-P, the method including: a step of esterifying dixylitol with acryloyl chloride to prepare dixylitol diacrylate (dXYA); and a step of reacting the dixylitol diacrylate with low molecular weight polyethylenimine (PEI) to obtain a polydixylitol polymer (PdXYP). The method further includes a step of coupling vitamin B6 to PdXYP. In the method, VBXYP-P is prepared by coupling TR-7 that is a cancer stem cell-targeting peptide to a polydixylitol polymer gene transporter (VB-VBXYP [VBXYP]) containing vitamin b6 using a Sulfo-SANPAH linker.

Specifically, the method for preparing a polydixylitol polymer gene transporter (VBXYP-P gene transporter:

Step a) of preparing dixylitol by an acetone/xylitol condensation method using xylitol and acetone;

Step b) of esterifying the dixylitol prepared in the step a) with acryloyl chloride to prepare dixylitol diacrylate (dXYA);

Step c) of performing a Michael addition reaction between the dixylitol diacrylate prepared in the step b) and low molecular weight polyethylenimine (PEI) to obtain a polydixylitol polymer (PdXYP);

Step d) of coupling vitamin B6 to the polydixylitol polymer (PdXYP) prepared in the step c); and

Step e) of coupling a cancer stem cell-targeting peptide (TR-7) to the polydixylitol polymer containing vitamin B (VBXYP) prepared in the step d),

Wherein the polydixylitol polymer gene transporter contains vitamin B6 and a TR-7 peptide.

As still another aspect, the present disclosure provides a nucleic acid delivery complex in which a therapeutic nucleic acid is bound to the VBXYP-P gene transporter.

The type of the therapeutic nucleic acid that may be complexed to VBXYP-P of the present disclosure is not particularly limited, and any nucleic acid that maybe delivered to a desired target and exhibit desired therapeutic effects according to the object of the present disclosure is included in the scope of the present disclosure. For example, the genes that may be delivered as a complex form with the polydixylitol polymer gene transporter of the present disclosure may include a normal gene of a disease-related therapeutic nucleic acid, a gene inhibiting expression of a target protein, large and small polynucleotides including antisense polynucleotides, and any RNA-type gene including ribozyme or siRNA. That is, the therapeutic nucleic acid of the present disclosure may be a plasmid form containing CRISPR sgRNA and Cas 9 genes, and may be selected from the group consisting of small interfering RNA (siRNA), small hairpin RNA (shRNA), endoribonuclease-prepared siRNAs (esiRNA) , antisense oligonucleotides, DNA, single-stranded RNA, double-stranded RNA, DNA-RNA hybrids, and ribozymes. The therapeutic nucleic acid of the present disclosure may be a nucleic acid for overexpressing or inhibiting genes that become specific causes of diseases, and in particular, the therapeutic nucleic acid of the present disclosure may be a nucleic acid corresponding to CRISPR sgRNA, small interfering RNA (siRNA), small hairpin RNA (shRNA), endoribonuclease-prepared siRNAs (esiRNA), and antisense oligonucleotides that inhibit expression of oncogene of cancer stem cells that is involved in cancer occurrence and recurrence, or may be a nucleic acid that induces expression of tumor suppressor genes that are involved in inhibition of cancer development or progression.

In particular, the therapeutic nucleic acid of the present disclosure may be a plasmid containing SMO CRISPR sgRNA (sequence: TATCGTGCCGGAAGAACTCC, SEQ ID NO:2, or AGGAGGTGCGTAACCGCATC, SEQ ID NO:3) for smoothened (SMO) that is a regulator of sonic hedgehog signaling, one of the self-renewal signaling of cancer stem cells, and cas9, siRNA, or esiRNA that is a complex mixture thereof, which may be SMO siRNA (esiRNA, Cat No: 4392420).

In order to effectively form the gene delivery complex according to the present disclosure, it is preferable to react the therapeutic nucleic acid with the VBXYP-P gene transporter at a molar ratio of 1:0.5 to 1:100, preferably 1:10 to 1:40, and more preferably 1:12 to 1:28.

The present inventors have conducted reactions of the VBXYP-P gene transporter with DNA at various molar ratios to examine a condensation capability for the therapeutic nucleic acid and a zeta potential of the VBXYP-P gene transporter according to the present disclosure. As a result, it was confirmed that when the molar ratio was 1:0.5 or more, the gene delivery complex (PdXYA/DNA) of the VBXYP-P gene transporter and DNA is most effectively formed (FIG. 4). It was confirmed that the nucleic acid delivery complex according to the present disclosure had an appropriate particle size to be used as a gene transporter because a relatively small and uniform particle size distribution of an average of 150 to 200 nm was exhibited (FIG. 5a), and the nucleic acid delivery complex according to the present disclosure was effectively bound to the anionic cell surface because a surface charge thereof exhibited a positive zeta potential of 25 to 40 mV (FIG. 5b).

The present inventors have observed the procedure of intracellular uptake into cancer stem cells and degradation of VBXYP-P and have confirmed cytotoxicity (FIGS. 6 and 7). It could be expected that cytotoxicity was low as exocytosis was induced by easy intracellular uptake and degradation of the gene transporter. As a result of the MTT assay, it was confirmed that the cytotoxicity of VBXYP-P was hardly exhibited compared to 25 kD PEI having high toxicity against cancer stem cells and glioblastoma multiforme cell lines.

In order to confirm the targeting ability of VBXYP-P to cancer stem cells, the present inventors have compared and assayed the gene delivery abilities of the representative non-viral gene transporters and VBXYP to which TR-7 was not coupled (FIG. 9). It was confirmed that only VBXYP-P exhibited a remarkably high gene delivery ability (about 60%) among various transporters to which green fluorescent protein genes were coupled.

The present inventors have confirmed whether apoptosis of cancer stem cells were induced when VBXYP-P to which SMO CRISPR (SMOcr) was complexed was delivered to the cancer stem cells. As a result, in the experimental group of the cancer stem cells treated with VBXYP-P/SMOcr, the lowest cell proliferative ability was confirmed (FIGS. 11 through 13), the fact that the apoptosis was occurring was confirmed (FIGS. 14 and 15), and it was demonstrated at the protein level that only the expression of SMO was inhibited, but the apoptosis was induced due to the inhibition of the expression of SMO as the expression of other proteins for signaling was also reduced (FIGS. 16 and 17).

The present inventors have confirmed whether apoptosis of cancer stem cells were induced when VBXYP-P to which SMO siRNA (siSMO) was complexed was delivered to the cancer stem cells. As a result, almost the same result as when the expression of SMO was inhibited using the CRISPR was confirmed. In the experimental group of the cancer stem cells treated with VBXYP-P/siSMO, the lowest cell proliferative ability was confirmed (FIGS. 18 through 20), the fact that the apoptosis was occurring was confirmed (FIGS. 21 and 22), and it was demonstrated at the protein level that only the expression of SMO was inhibited, but the apoptosis was induced due to the inhibition of the expression of SMO as the expression of other proteins of signaling was also reduced (FIGS. 23 and 24).

Finally, in order to confirm whether VBXYP-P permeates the blood brain barrier to target cancer stem cells in glioblastoma multiforme and thus to deliver genes, the research team has conducted an experiment using a 3D microfluidic system in vitro (FIGS. 25 through 27). As a result, it was confirmed that VBXYP-P has permeated BBB, and after permeating BBB, VBXYP-P targeted cancer stem cells present in glioblastoma multiforme in significantly small amounts, and thus, genes were delivered.

As another aspect, the present disclosure provides a pharmaceutical composition for gene therapy containing, as an active ingredient, the nucleic acid delivery complex in which a therapeutic nucleic acid is complexed to VBXYP-P. The pharmaceutical composition of the present disclosure may be used for a treatment or prevention of treatable diseases with gene therapy depending on the type of therapeutic nucleic acid constituting the pharmaceutical composition.

The pharmaceutical composition of the present disclosure maybe administered with a pharmaceutically acceptable carrier, and when the pharmaceutical composition is administered orally, in addition to the active ingredient, a binder, a lubricant, a disintegrant, an excipient, a solubilizer, a dispersant, a stabilizer, a suspending agent, a pigment, a flavoring agent, and the like maybe further included. In a case of an injection, the pharmaceutical composition of the present disclosure may be used by being mixed with a buffer, a preserving agent, a painkiller, a solubilizer, an isotonic agent, a stabilizer, and the like. In addition, when the composition of the present disclosure is topically administered, a base material, an excipient, a lubricant, a preserving agent, and the like may be used.

The composition of the present disclosure may be prepared in various formulations after being mixed with the pharmaceutically acceptable carrier as described above, and in particular, the composition of the present disclosure may be prepared in a formulation for inhalation administration or injection administration. For example, when the composition of the present disclosure is administered orally, the composition may be prepared in a form of a tablet, a troche, a capsule, an elixir, a suspension, a syrup, a wafer, or the like. In a case of an injection, the composition of the present disclosure maybe prepared in a unit dose ampoule or a multiple dose administration type. The composition of the present disclosure maybe formulated into other solutions, suspensions, tablets, pills, capsules, sustained-release preparations, and the like. The drug delivery by inhalation is one of the non-invasive methods, and in particular, the delivery of the therapeutic nucleic acid by an inhalation formulation (for example, aerosol) may be effectively used for a wide range of treatments for lung diseases. This is because the anatomical structure and location of the lungs allow immediate and non-invasive approaches and may receive the topical application of the gene delivery system without affecting other organs.

Meanwhile, examples of a carrier, an excipient, and a diluent applicable for formulations may include lactose, dextrose, sucrose, sorbitol, mannitol, xylitol, erythritol, maltitol, starch, acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. In addition, a filler, an anti-cohesive agent, a lubricant, a wetting agent, a flavoring agent, a preservative, and the like may be further included.

The pharmaceutical composition of the present disclosure may be administered orally or parenterally. Examples of an administration route of the pharmaceutical composition of the present disclosure may include, but are not limited to, oral, intravenous, intramuscular, intra-arterial, intramedullary, intradural, intracardiac, percutaneous, subcutaneous, intraperitoneal, enteral, hypoglossal, and topical administrations. For such clinical administrations, the pharmaceutical composition of the present disclosure may be formulated into proper formulations using a known technique. For example, when the pharmaceutical composition is administered orally, the pharmaceutical composition may be administered by being mixed with an inert diluent or an edible carrier, sealed in a hard or soft gelatin capsule, or compressed into a tablet. In the case of oral administration, active ingredients may be mixed with an excipient and then used as a form of a tablet for ingestion, a buccal tablet, a troche, a capsule, an elixir, a suspension, a syrup, a wafer, or the like. In addition, various formulations for injections, parenteral administrations, and the like may be prepared by known methods or conventional methods in the art.

An effective administration dose of the pharmaceutical composition of the present disclosure has a wide range depending on a weight, age, sex, health conditions, diet of a patient, an administration time, an administration method, an excretion rate, and severity of diseases, and may be readily determined by those skilled in the art.

As for the pharmaceutical composition of the present disclosure, the therapeutic nucleic acid constituting the pharmaceutical composition and the targeting peptide (TR-7) contained in the transporter may inhibit expression of smoothened (SMO) in cancer stem cells to target the cancer stem cells, thereby inducing apoptosis of the cancer stem cells. The therapeutic nucleic acid may be a plasmid containing SMO CRISPR sgRNA (sequence: TATCGTGCCGGAAGAACTCC, SEQ ID NO:2, or AGGAGGTGCGTAACCGCATC, SEQ ID NO:3) and cas9, or SMO siRNA (esiRNA, Cat No: 4392420). The pharmaceutical composition of the present disclosure may have an effect of treating or preventing cancer stem cells according to the type of the therapeutic nucleic acid constituting the pharmaceutical composition. The cancer may be selected from the group consisting of a lung cancer, a bone cancer, a pancreatic cancer, a skin cancer, a head and neck cancer, skin melanoma, a uterine cancer, an ovarian cancer, a rectal cancer, a colorectal cancer, a colon cancer, a breast cancer, uterine sarcoma, fallopian tube carcinoma, endometrial carcinoma, cervix carcinoma, vagina carcinoma, vulva carcinoma, an esophageal cancer, a small intestine cancer, a thyroid cancer, a parathyroid cancer, soft tissue sarcoma, a urethral cancer, a penile cancer, a prostate cancer, chronic or acute leukemia, a pediatric solid tumor, differentiated lymphoma, a bladder cancer, a kidney cancer, renal cell carcinoma, renal pelvic carcinoma, primary central nervous system lymphoma, a myelencephalon tumor, brain stem glioma, and pituitary gland adenoma.

As further still another aspect, the present disclosure provides cancer stem cell gene therapy using the polydixylitol polymer gene transporter of the present disclosure, the nucleic acid delivery complex containing the transporter, and the pharmaceutical composition containing the complex described above.

### Advantageous Effects

As set forth above, according to an exemplary embodiment in the present disclosure, it is confirmed that the polydixylitol polymer gene transporter (VBXYP-P) to which vitamin B6 and a cancer stem cell-specific peptide are coupled has a remarkably higher nucleic acid delivery rate to cancer stem cells than the pre-existing nucleic acid transporter, has almost no cytotoxicity in a complex when electrostatically bound with DNA, and above all, permeates a blood brain barrier to target cancer stem cells in glioblastoma multiforme and thus to specifically deliver the nucleic acid, thereby inducing transformation. Therefore, the gene transporter of the present disclosure inhibits expression of cancer stem cells in a tumor in vivo, and thus may be broadly used in gene therapy for various cancer diseases.

### DESCRIPTION OF DRAWINGS

FIG. 1 is a view illustrating a procedure of synthesizing a polydixylitol polymer gene transporter (PdXYA) of the present disclosure to be used as an initial backbone.

FIG. 2 is a view illustrating a procedure of synthesizing a vitamin B6-coupled polydixylitol polymer gene transporter (VB-PdXYA) of the present disclosure to be used as a backbone.

FIG. 3a is a view illustrating a procedure of synthesizing Sulfo-SANPAH and a transporter in order to couple TR-7 (SEQ ID NO:1) that is a cancer stem cell target marker to the gene transporter of the present disclosure.

FIG. 3b is a view illustrating a procedure of synthesizing TR-7 (SEQ ID NO:1) that is a cancer stem cell target marker with the Sulfo-SANPAH-coupled transporter of the present disclosure.

FIG. 4 illustrates a result of a gel retardation experiment for an ability to form a polyplex by electrostatically binding siRNA or pDNA to VBXYP-P that is the polymer gene transporter of the present disclosure, and is a view illustrating a result of gel electrophoresis on a PdXYP-P/siRNA polyplex formed by reacting VBXYP-P with siRNA at molar ratios (N/P) of 0.05, 0.1, 0.3, 0.5, and 1.0.

FIG. 5 illustrates results of comparing sizes and zeta potentials of VBXYP used as the backbone of the polymer gene transporter of the present disclosure and VBXYP-P to which TR-7 is coupled.

FIG. 6 illustrates a procedure of intracellular uptake and degradation of VBXYP-P of the present disclosure and a green fluorescent protein gene (tGFP plasmid) complex in cancer stem cells.

FIG. 7 is a view illustrating results of evaluating cytotoxicity of VBXYP-P of the present disclosure in vitro in cancer stem cells (CSCs) and glioblastoma multiforme (GBM) by an MTT assay and comparing the results with those of PEI 25 kDa and VBXYP.

FIG. 8 is a view illustrating results of comparison of expression of fluorescence of cancer stem cells and glioblastoma multiforme cell lines treated with a VBXYP/DNA polyplex formed by reacting VBXYP-P of the present disclosure with DNA at various weight ratios (w/w) (2:1, 4:1, 6:1, 8:1, 10:1, and 20:1).

FIG. 9 is a view illustrating results of comparison of expression of fluorescence of cancer stem cells and glioblastoma multiforme cell lines treated with nucleic acid delivery complexes obtained by binding a green fluorescent protein gene (tGFP gene) to each of the gene transporter and various other gene transporters (lipofectamin, PEI 25 kD, and VBPEA) in order to examine transformation efficiency of the VBXYP-P gene transporter of the present disclosure.

FIG. 10 illustrates results of comparative assay of a degree of cancer stem cell targeting of VBXYP-P of the present disclosure using a fluorescence-activated cell sorting flow cytometry (FACS).

FIG. 11 illustrates results of a cell live/dead assay to examine that when a polyplex of VBXYP-P of the present disclosure and a smoothened (SMO) CRISPR-cas9 plasmid is delivered to cancer stem cells, how it affects proliferation of the cancer stem cells.

FIG. 12 is a view illustrating comparison of differences in cell proliferation evaluated in cancer stem cells (CSCs) by a WST-1 assay when a polyplex of VBXYP-P of the present disclosure and SMO CRISPR (SMOcr) is delivered to the cancer stem cells.

FIG. 13 is a view illustrating comparison of the degrees of cell proliferation evaluated by a 5-ethynyl-2′-deoxyuridine (EdU) cell proliferation assay when a polyplex of VBXYP-P of the present disclosure and SMO CRISPR (SMOcr) is delivered to the cancer stem cells.

FIG. 14 illustrates results of evaluating apoptosis by a TUNEL assay after SMO CRISPR is delivered to cancer stem cells using VBXYP-P of the present disclosure.

FIG. 15 illustrates results of evaluating apoptosis by an Annexin V assay after SMO CRISPR is delivered to cancer stem cells using VBXYP-P of the present disclosure.

FIG. 16 illustrates results of evaluating and assaying a change in expression of each of smoothened (SMO) and sonic hedgehog (Shh) by immunocytochemistry staining (ICC staining) after SMO CRISPR is delivered to cancer stem cells using VBXYP-P of the present disclosure.

FIG. 17 illustrates results of evaluating and assaying a change in expression of each of smoothened (SMO) and sonic hedgehog (Shh) by a western blot protein quantitative assay after SMO CRISPR is delivered to cancer stem cells using VBXYP-P of the present disclosure.

FIG. 18 illustrates results of a cell live/dead assay to examine that when a polyplex of VBXYP-P of the present disclosure and smoothened (SMO) siRNA is delivered to cancer stem cells, how it affects proliferation of the cancer stem cells.

FIG. 19 is a view illustrating comparison of differences in cell proliferation evaluated in cancer stem cells (CSCs) by a WST-1 assay when a polyplex of VBXYP-P of the present disclosure and SMO siRNA is delivered to the cancer stem cells.

FIG. 20 is a view illustrating comparison of the degrees of cell proliferation evaluated by a 5-ethynyl-2′-deoxyuridine (EdU) cell proliferation assay when a polyplex of VBXYP-P of the present disclosure and SMO siRNA (siSMO) is delivered to the cancer stem cells.

FIG. 21 illustrates results of evaluating apoptosis by a TUNEL assay after SMO siRNA is delivered to cancer stem cells using VBXYP-P of the present disclosure.

FIG. 22 illustrates results of evaluating apoptosis by an Annexin V assay after SMO siRNA is delivered to cancer stem cells using VBXYP-P of the present disclosure.

FIG. 23 illustrates results of evaluating and assaying a change in expression of each of smoothened (SMO) and sonic hedgehog (Shh) by immunocytochemistry staining (ICC staining) after SMO siRNA is delivered to cancer stem cells using VBXYP-P of the present disclosure.

FIG. 24 illustrates results of evaluating a change in expression of each of smoothened (SMO) and sonic hedgehog (Shh) by a western blot protein quantitative assay after SMO siRNA is delivered to cancer stem cells using VBXYP-P of the present disclosure.

FIG. 25 illustrates results of observing whether VBXYP-P and VBXYP of the present disclosure permeate a blood brain barrier (BBB) using a microfluidic chip having the inside in which brain astrocytes are cultured and the outer passage in which human umbilical vein endothelial cells (HUVECs) are cultured and simulating a three-dimensional BBB structure in vitro, and illustrates results of qualitatively assaying the degree of transfection by delivery of tGFP to cells inside BBB.

FIG. 26 illustrates results of comparative assay of whether a polyplex of VBXYP-P and tGFP permeates BBB and whether genes are delivered to cancer stem cells after a BBB model is formed using the three-dimensional microfluidic BBB model chip having the inside in which only cancer stem cells are cultured and the outer passage in which HUVECs are cultured.

FIG. 27 illustrates results of comparative assay of whether VBXYPd and VBXYP-P deliver genes targeting cancer stem cells in glioblastoma multiforme after a BBB model is formed using the three-dimensional microfluidic BBB model chip having the inside in which only glioblastoma multiforme cell lines are cultured and the outer passage in which HUVECs are cultured.

### BEST MODE FOR INVENTION

As an exemplary embodiment, the present disclosure provides a gene transporter (VBXYP-P) to which vitamin B6 is coupled using the previously invented polydixylitol polymer (PdXYP) (Chemical Formula 3) as an initial backbone and in which a peptide (TR-7 peptide) binding specifically to a CD133 protein that is a marker for cancer stem cells is contained. The present disclosure is designed to target cancer stem cells and thus to deliver genes by improving the polydixylitol polymer gene transporters (PdXYP and VB-PdXYP (VBXYP)) that are the previously developed gene transporters. The gene transporter of the present disclosure may have a structure of the following Chemical Formula 1.

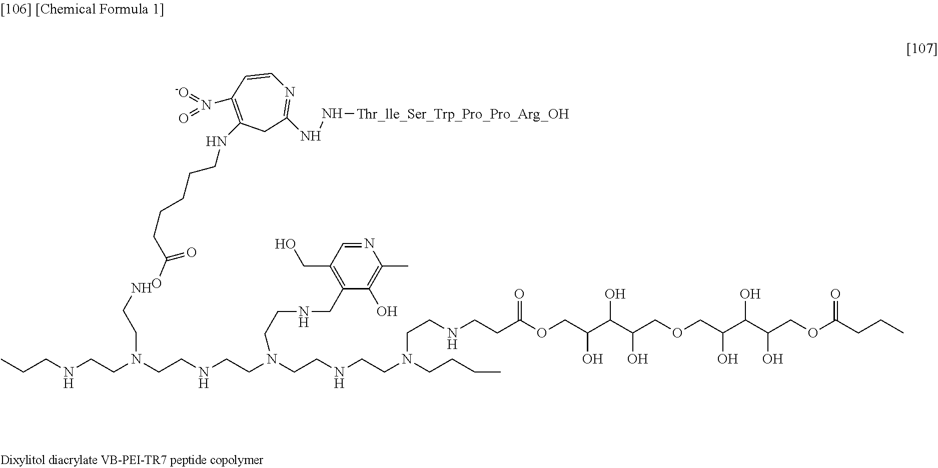

Sulfosuccinimidyl-6-[4′-azido-2′-nitrophenylamino]hex anoate (sulfo-SANPAH) has a structure of Chemical Formula 2. A polydixylitol polymer gene transporter (dixylitol diacrylate VB-PEI-TR7 peptide copolymer, VBXYP-P) in which a cancer stem cell-specific peptide (TR-7 peptide, SEQ ID NO:1) is coupled to the previously developed VB-PdXYP (VBXYP) gene transporter using this linker was prepared.

### Mode for Invention

Hereinafter, the present disclosure will be described in more detail with reference to Inventive Examples. These Inventive Examples are only to illustrate the present disclosure and are not to be interpreted that the scope of the present disclosure is limited to these Inventive Examples.

### Inventive Example 1: Used Reagents and Materials

In the present disclosure, a polydixylitol polymer gene transporter (VBXYP-P) in which vitamin B6 was contained and to which TR-7 that was a cancer stem cell-specific peptide was coupled was prepared, and the following materials and reagents were used to confirm the following Inventive Examples.

Products manufactured by Sigma-Aldrich (St. Louis, Mo., USA) were used as reagents such as branched poly(ester imine) (bPEI) (Mn: 1.2 k and 25 k), dimethyl sulfoxide (DMSO), acryloyl chloride, xylitol, pyridoxal 5-phosphate (PLP), 4′-deoxypyridoxine hydrochloride, sodium cyanoborohydride (NaCNBH4), genistein, chlorpromazine, bafilomycin A1, 3-(4,5-dimethylthiazol-2-yl)-2,5-diphenyltetrazolium bromide (MTT), and
sulfosuccinimidyl-6-[4′-azido-2′-nitrophenylamino]hexanoate (Sulfo-SANPAH). TR-7 that was a peptide to which CD133, which was a cancer stem cell marker, was bound was synthesized through A&PEP Inc. In addition, Luciferase reporter coding for firefly (Photonus pyralis), a pGL3-vector, and an enhancer were obtained from Promega Corporation (Madison, Wis., USA). Green fluorescent protein (GFP) genes were obtained from Clontech Laboratories, Inc. (Palo Alto, Calif., USA). Tetramethylrhodamine isothiocyanate (TRITC) and YOYO-1 iodide (Molecular Probes, Invitrogen, Oregon, USA) dyes were used for confocal microscope analysis. Scrambled siRNA (siScr) was obtained from Genolution Pharmaceuticals Inc. (Republic of Korea), and smoothened siRNA (siSMO) was obtained from Thermo Fisher Scientific (USA). In addition, smoothened SMO CRISPR (SMOcr) was obtained from GenScript Biotech Corp. (USA). Finally, a 3D BBB microfluidic chip was obtained from SynVivo, Inc. (USA).

### Inventive Example 2: Preparation of Polyol-Based Osmotic Polydixylitol Polymer Gene Transporter to which Vitamin B6 and TR-7 Peptide are Coupled

A polyol-based osmotic polydixylitol polymer gene transporter (VBXYP-P) to which vitamin B6 and a TR-7 peptide were coupled according to the present disclosure was synthesized by the following five steps. The gene transporter of the present disclosure was invented by improving the patent materials previously invented by the inventors. Therefore, the registered patent (10-1809795) was cited up to the step 4.

2-1. Synthesis of Dixylitol

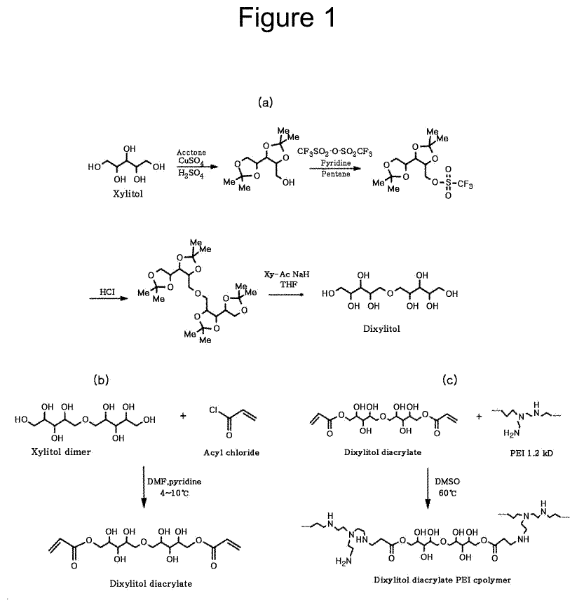

The present inventors have noticed that the number and stereochemistry of hydroxy groups affect the intracellular delivery, and thus, have tried to develop materials for gene delivery having enhanced delivery efficiency into the cells by controlling osmotically active hydroxy groups. Since sugar alcohols having 8 hydroxy groups were not commercially available, the present inventors have directly synthesized a xylitol dimer and dixylitol as an analogue of an octamer by the procedure illustrated in FIG. 1.

Specifically, xylitol was first crystallized into diacetone xylitol (Xy-Ac) crystals by an acetone/xylitol condensation method of Raymond and Hudson. The terminal hydroxy group of diacetone xylitol was reacted with trifluoromethyl sulfonyl chloride (CF₃SO₂—O—SO₂CF₃) to produce trifluoromethane sulfonyl xylitol (TMSDX). The prepared trifluoromethane sulfonyl xylitol was reacted in the same molar amount of diacetone xylitol in the presence of dry THF to form dixylitol diacetone (Xy-Ac dimer). The reaction product was finally converted into the xylitol dimer by opening the chemical rings in a HCl/MeOH solution ((a) of FIG. 1).

2-2. Synthesis of Dixylitol Diacrylate

A dixylitol diacrylate (dXYA) monomer was synthesized by esterifying dixylitol with 2 equivalents of acryloyl chloride. An emulsion was prepared by dissolving dixylitol (1 g) in DMF (20 ml) and pyridine (10 ml) and adding dropwise an acryloyl chloride solution (1.2 ml dissolved in 5 ml of DMF) to the mixture at 4° C. while constantly stirring the mixture. After completion of the reaction, the HCl-pyridine salt was filtered and the filtrate was added dropwise to diethyl ether. The product was precipitated with a syrup liquid and dried under vacuum.

2-3. Synthesis of Polydixylitol Polymer (PdXYP)

The polydixylitol polymer (PdXYP) of the present disclosure was prepared by a Michael addition reaction between low molecular weight polyethyleneimide (bPEI) (1.2 k) and dixylitol diacrylate (dXYA).

Specifically, the synthesized dXYA (0.38 g) dissolved in DMSO (5 ml) was added dropwise to 1 equivalent of bPEI (1.2 kDa, dissolved in 10 ml of DMSO) and reacted at 60° C. while constantly stirring the mixture for 24 hours. After completion of the reaction, the mixture was dialyzed with distilled water using a Spectra/Por membrane (MWCO: 3,500 Da; Spectrum Medical Industries, Inc., Los Angeles, Calif., USA) at 4° C. for 36 hours.

Finally, the synthesized polymer was lyophilized and stored at −70° C.

2-4. Synthesis of Vitamin B6-Coupled Polydixylitol Polymer Gene Transporter (VB-PdXYP or VBXYP)

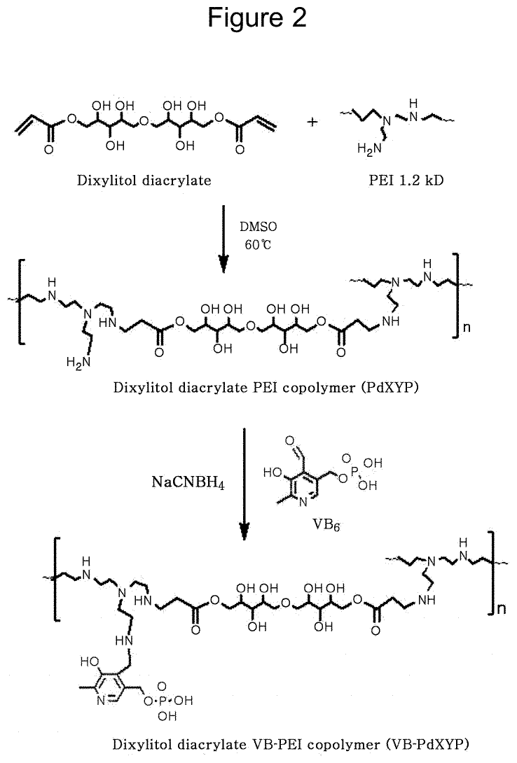

Pyridoxal 5′phosphate (PLP) and a polydixylitol polymer gene transporter (PdXYP) were reacted with each other to form a transient Schiff base. Thereafter, the Schiff base was reduced using NaCNBH₄ to obtain a vitamin B6-coupled polydixylitol polymer gene transporter (VB-PdXYP or VBXYP) (FIG. 2).

2-5. Synthesis of Polydixylitol Polymer Gene Transporter (VBXYP-P) to which Vitamin B6 and TR-7 are Coupled

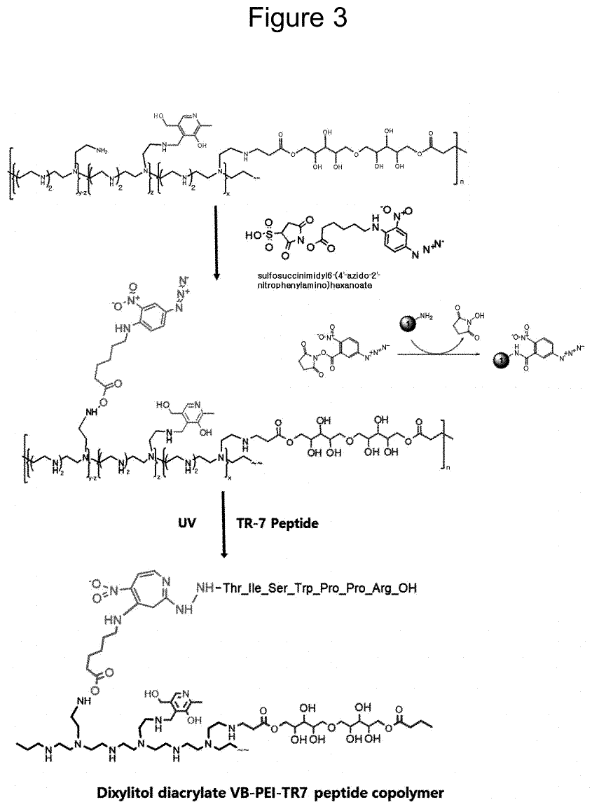

N-Hydroxysuccinimide (NHS) of Sulfo-SANPAH formed a stable amide bond with a primary amine group of low molecular weight polyethylenimine (PEI) of the VBXYP gene transporter in a buffer solution environment with a pH 7 to 9, and nitrophenyl azide was bound to an amine group of TR-7 that was a cancer stem cell-specific peptide through a dehydroazepine intermediate by a reaction of an ultraviolet light of 300 to 460 nm. As a result, VBXYP-P was obtained (FIG. 3).

### Inventive Example 3: Analysis of Properties of Polymer Gene Transporter

3-1. Formation of Polymer Gene Transporter Nanoplex (VBXYP-P Nanoplex)

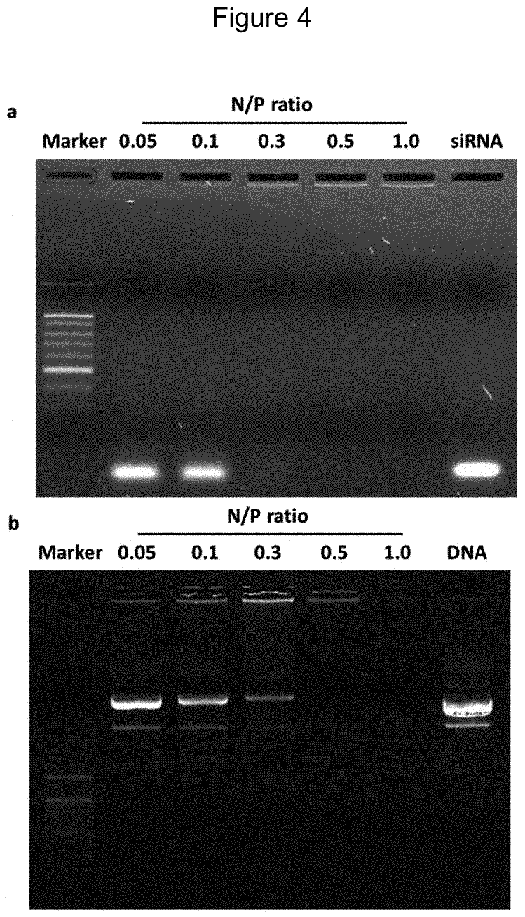

An ability of VBXYP-P of the present disclosure to form a polyplex by being bound with pDNA or siRNA was confirmed by a gel retardation experiment. Specifically, the gel retardation experiment was conducted by gel electrophoresis on a VBXYP-P/pDNA or VBXYP-P/siRNA polyplex produced by reacting PdXYP with pDNA or siRNA at molar ratios (N/P) of 0.05, 0.1, 0.3, 0.5, and 1.0. As a result, in the case of VBXYP-P/siRNA, a polyplex was formed well at molar ratios (N/P) of 0.3, 0.5, and 1 (FIG. 4a), and in the case of VBXYP-P/pDNA, a polyplex was formed well at molar ratios (N/P) of 0.5 and 1 (FIG. 4b).

3-2. Size and Zeta Potential of Polymer Gene Transporter Nanoplex (VBXYP-P Nnoplex)

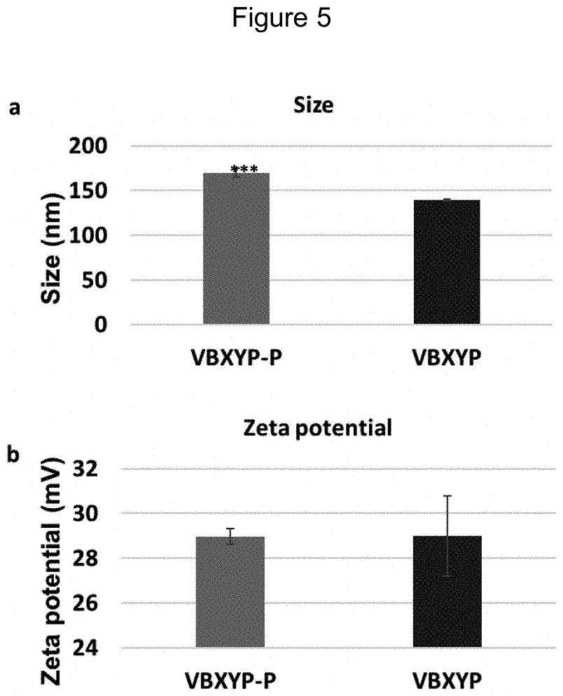

Sizes and zeta potentials of VBXYP-P of the present disclosure and VBXYP previously invented were compared and analyzed using a dynamic light scattering apparatus (FIG. 5). Asa result, the size of VBXYP-P was greater than that of VBXYP, and the zeta potential of VBXYP was greater than or similar to that of VBXYP-P. Theoretically, the zeta potential is reduced because the TR-7 peptide is bound to the amine group of PdXYP.

3-3. Evaluation of Intracellular Uptake and Cytotoxicity of Polymer Gene Transporter Nanoplex (VBXYP-P Nanoplex)

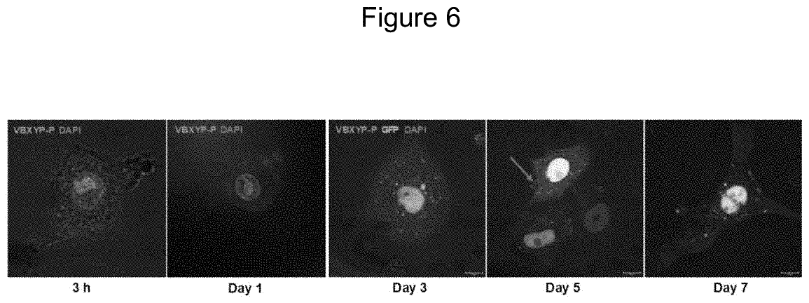

FIG. 6 illustrates a procedure of intracellular uptake and degradation of VBXYP-P. After TRITC exhibiting red fluorescence was tagged to the VBXYP-P gene transporter, a polyplex was formed with a green fluorescent protein gene, cancer stem cells were treated with the polyplex, and changes were observed for 7 days. As a result, after 3 hours, VBXYP-P exhibiting red fluorescence was found throughout the cells. However, it was confirmed that the red fluorescence gradually disappeared and green fluorescence was expressed a lot in the cells until 7 days passed. This means that intracellular uptake of the transporter into the cancer stem cells is excellent, the gene delivery is excellent, and the transporter is degraded and does not remain in the cells. As such, when the transporter is degraded well and extracellularly released, it may be expected that cytotoxicity will be reduced.

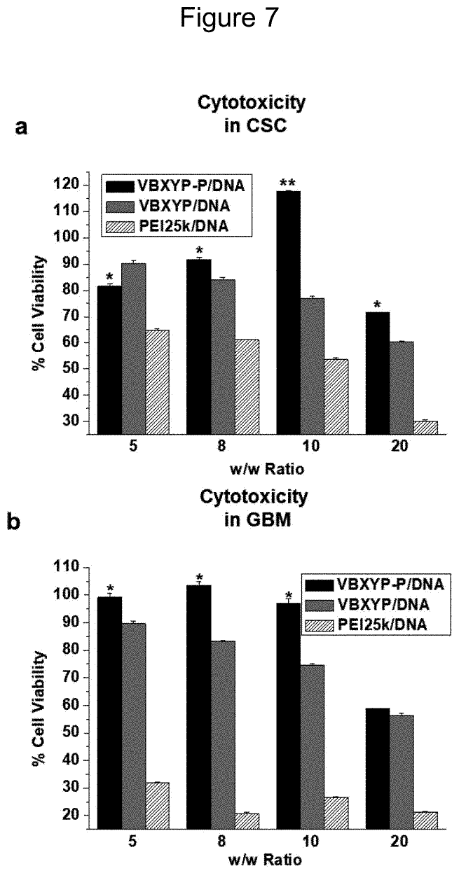

FIG. 7 illustrates results of assay of cytotoxicity of VBXYP-P against cancer stem cells and glioblastoma multiforme cell lines. The cytotoxicity of 25 kD PEI generally used for gene delivery was compared with the cytotoxicity of VBXYP previously invented. As a result, it was confirmed that the cytotoxicity of VBXYP-P was hardly exhibited compared to 25 kD PEI exhibiting high cytotoxicity.

### Inventive Example 4: Cancer Stem Cell Targeting of Polyol-Based Osmotic Polydixylitol Polymer Gene Transporter (VBXYP-P) to which Vitamin B6 and TR-7 Peptide are Coupled

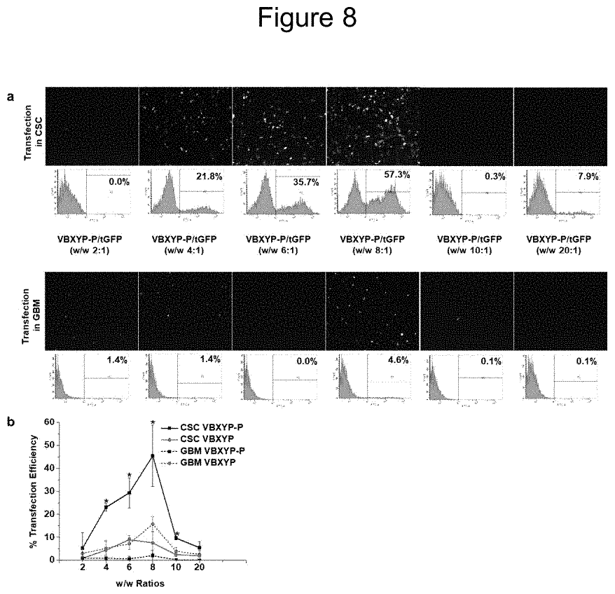

As a result of confirming a ratio (w/w) of VBXYP-P having the optimal gene delivery rate to cancer stem cells to genes, the polyplex prepared at the ratio of 8:1 had the highest gene delivery ability (FIG. 8).

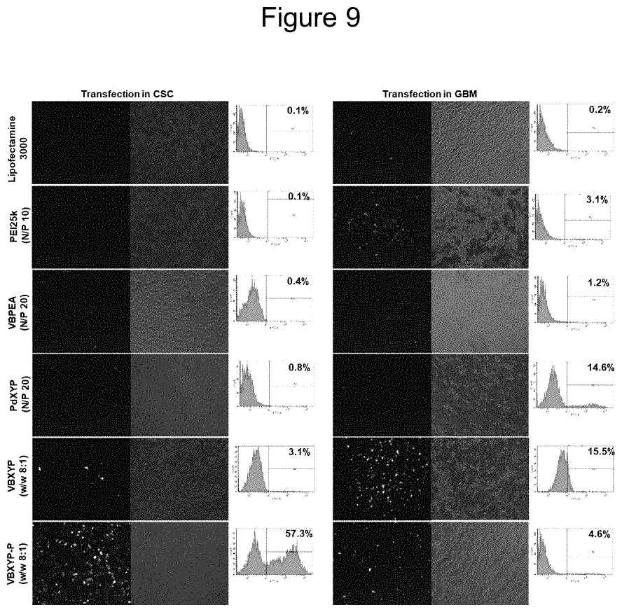

In addition, in order to confirm the cancer stem cell targeting gene delivery ability of VBXYP-P, the gene delivery efficiency of VBXYP-P was compared with the gene delivery efficiency of each of the gene transporters previously invented by the inventors and various commercially available non-viral gene transporters (Lipofectamine 3000, 25 kD PEI, VBPEA, PdXYP, and VBXYP) (FIG. 9). Asa result, only the VBXYP-P transporter induced transfection of the cancer stem cells with remarkably high efficiency. VBXYP to which TR-7 that was a cancer stem cell-targeting peptide was not coupled induced about 3% of transfection, but VBXYP-P induced about 60% of transfection. It was confirmed from these results that TR-7 significantly acted on targeting of the cancer stem cells.

In addition, a very interesting result was confirmed. In the case of VBXYP to which TR-7 was not coupled, the transfection efficiency for the cancer stem cells was low, but the transformation efficiency for the glioblastoma multiforme cell lines was the highest compared to other transporters. It was confirmed that the excellent gene delivery abilities to cancer stem cells exhibited using the vitamin B6-coupled gene transporters, which have been confirmed in the previous patents (10-2015-0014399 and 10-1809795), were equally applied to other cell lines. Cancer tissues consume a high amount of vitamin B6 and thus have a high uptake rate of extracellular vitamin B6. Therefore, the vitamin B6-coupled gene transporter may have a gene delivery ability specific to cancer tissues. VBXYP-P also contains vitamin B6. However, a gene delivery efficiency of less than 5% with respect to glioblastoma multiforme cell lines was confirmed. From this, it is possible to assume that the targeted delivery by TR-7 is a higher priority than the gene delivery by the vitamin B6.

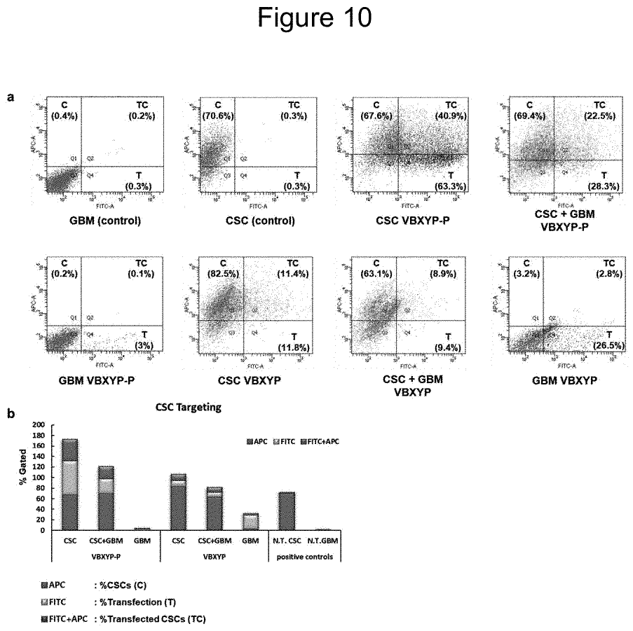

In order to more accurately confirm the cancer stem cell targeting gene delivery ability of VBXYP-P, cancer stem cells were labeled with CD133 antibodies to which allophycocyanin (APC) was bound, the cancer stem cells were treated with VBXYP-P/GFP, and then, comparison of targeting abilities was conducted by an FACS assay (FIG. 10). As a result, it was confirmed that the targeting ability of VBXYP-P was remarkably higher than that of VBXYP with no TR-7.

### Inventive Example 5: Induction of Apoptosis by Induction of Knock-Out of Smoothened (SMO) in Cancer Stem Cells Using VBXYP-P and CRISPR-cas9 System

5-1. Change in Cell Proliferation of Cancer Stem Cells by Smoothened CRISPR (SMOcr) Delivery

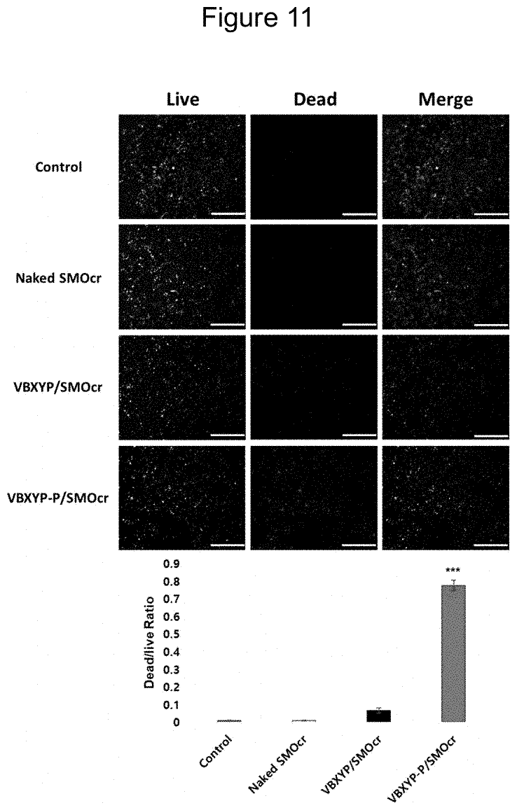

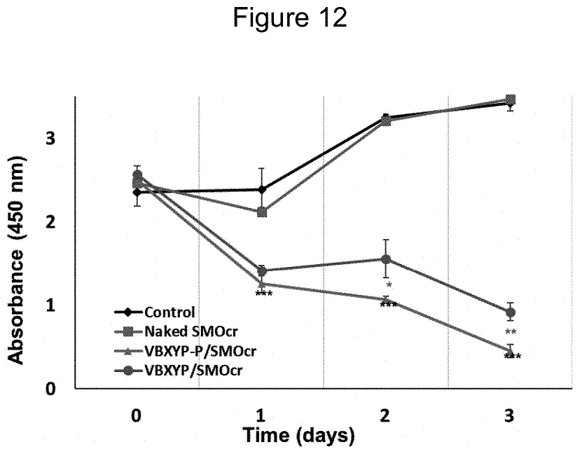

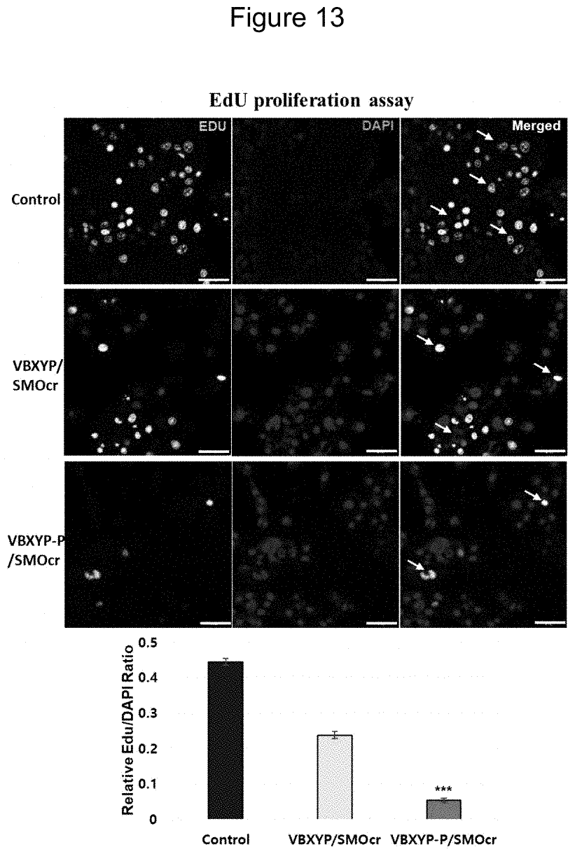

First, a chance in proliferative ability of cancer stem cells after treatment of VBXYP-P/SMOcr was confirmed by a cell live/dead assay (FIG. 11). Living cells exhibit green fluorescence, and dying cells exhibit red fluorescence. As a result of the experiment, it was confirmed that the percentage of dying cells was the highest in the cancer stem cell group treated with VBXYP-P/SMOcr. In addition, in the WST-1 proliferation evaluation, the same results were obtained significantly in the experimental group treated with VBXYP-P/SMOcr (FIG. 12). Finally, the proliferative ability of SMOcr delivery on the cancer stem cells was confirmed by an EdU assay in which fluorescence was exhibited by coupling of EdU to newly synthesized genes (FIG. 13). As a result, as with the above experimental results, the lowest fluorescence was exhibited in the experimental group treated with VBXYP-P/SMOcr. From these results, it was confirmed that SMOcr delivery induced a significant reduction in proliferative ability of the cancer stem cells, and the hypothesis that the SMOcr delivery induces apoptosis of the cancer stem cells was established.

5-2. Confirmation of Induction of Apoptosis of Cancer Stem Cells by Smoothened CRISPR (SMOcr) Delivery

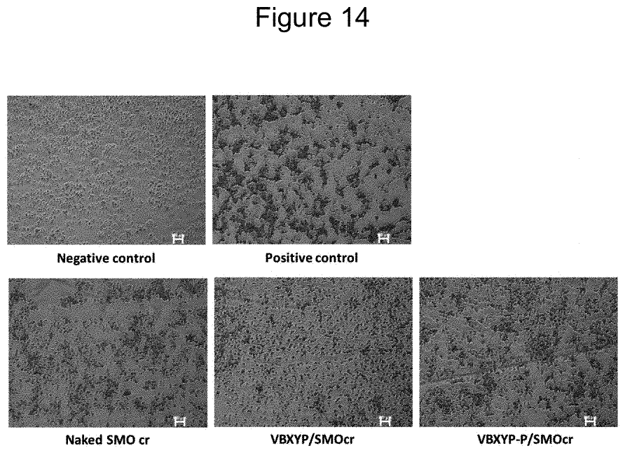

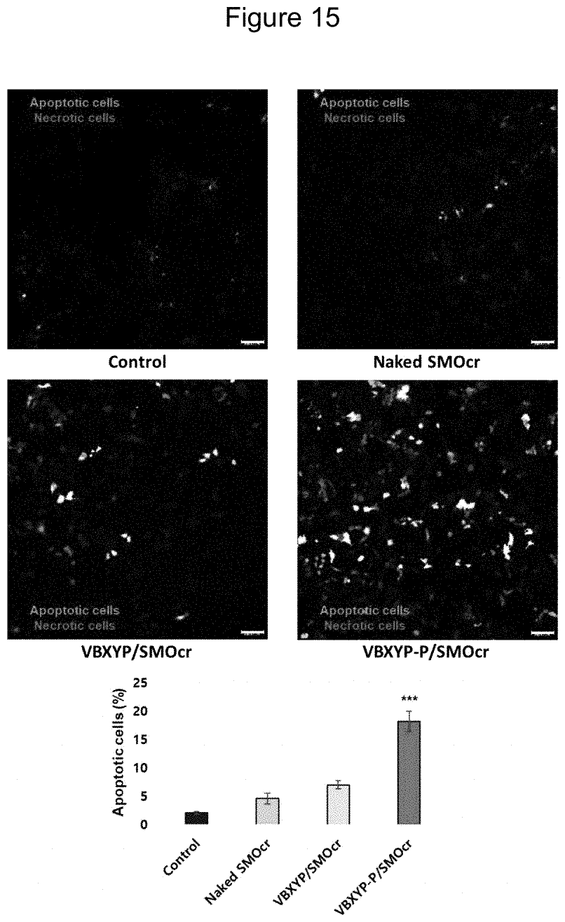

In order to confirm whether apoptosis of cancer stem cells were induced by SMOcr delivery, a TUNEL assay and an Annexin V assay were performed (FIGS. 14 and 15). A colorimetric tunel assay was employed in the TUNEL assay. This assay is an assay to easily observe apoptotic cells with an optical microscope by coupling uridine triphosphate (UTP) to the 3′ terminus of broken DNA using terminal deoxynucleotidyl transferase (TdT) and staining DNA to exhibit dark brown. As a result of the experiment, in the experimental group of the cancer stem cells treated with VBXYP-P/SMOcr, the dark brown color was most observed.

In the Annexin V assay, phosphatidylserine inside cells is extracellularly exposed due to disruption of a cell membrane structure at the early stage of apoptosis, and Annexin V binds to the phosphatidylserine to exhibit fluorescence, such that the early stage of apoptosis is confirmed. As with the results of the TUNEL assay, in the results of this assay, in the experimental group treated with VBXYP-P/SMOcr, the fluorescence was most exhibited. It was demonstrated through these results that apoptosis of the cancer stem cells were induced by knock-out of SMO in the cancer stem cells using SMOcr.

5-3. Assay of Expression of Smoothened after Knock-Out of Smoothened

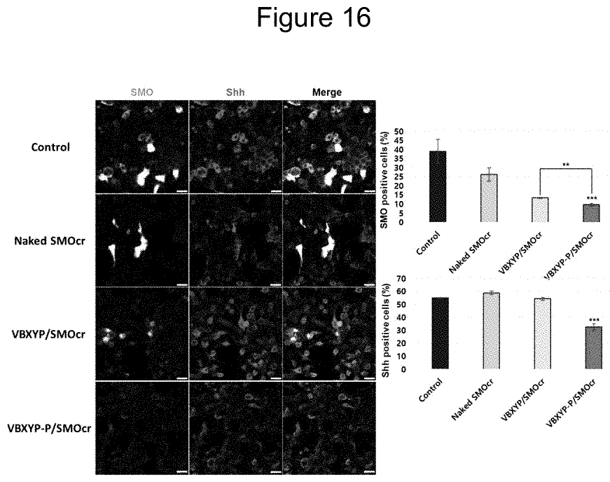

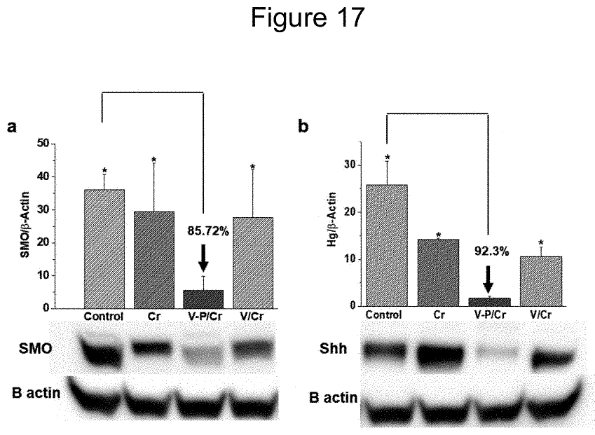

Knock-out of SMO was induced by SMOcr delivery to cancer stem cells using VBXYP-P of the present disclosure, and a change in expression of SMO was assayed by immunofluorescence (FIG. 16). As a result, in the cancer stem cells treated with VBXYP-P/SMOcr, expression of each of SMO (green) and sonic hedgehog (Shh) was the lowest compared to those in other experimental groups, and in the results of the western blot protein quantitative assay, the same results were confirmed (FIG. 17). The amount of SMO was reduced by about 86% compared to the control group, and the amount of Shh was reduced by about 92% compared to the control group. Shh is released from dispatch protein in an autocraine or paracrine manner, Shh may be incompletely released from cells undergoing apoptosis through inhibition of the expression of SMO, and thus, the amount of Shh may be naturally reduced. Shh is an important protein that initiates a sonic hedgehog signaling inducing self-renewal of cancer stem cells. However, the expression of Shh is reduced by knock-out of SMO, and accordingly, a self-renewal ability of the cancer stem cells is reduced, such that apoptosis of the cancer stem cells may be accelerated.

Based on these results, the present inventors have demonstrated that inhibition of expression of SMO in cancer stem cells treated with VBXYP-P/SMOcr induced a reduction in expression of Shh, and apoptosis of the cancer stem cells could be induced by disruption of the self-renewal pathways.

### Inventive Example 6: Induction of Apoptosis by Induction of Knock-Down of Smoothened (SMO) in Cancer Stem Cells Using VBXYP-P and siRNA

6-1. Change in Cell Proliferation of Cancer Stem Cells by Smoothened siRNA (siSMO) Delivery

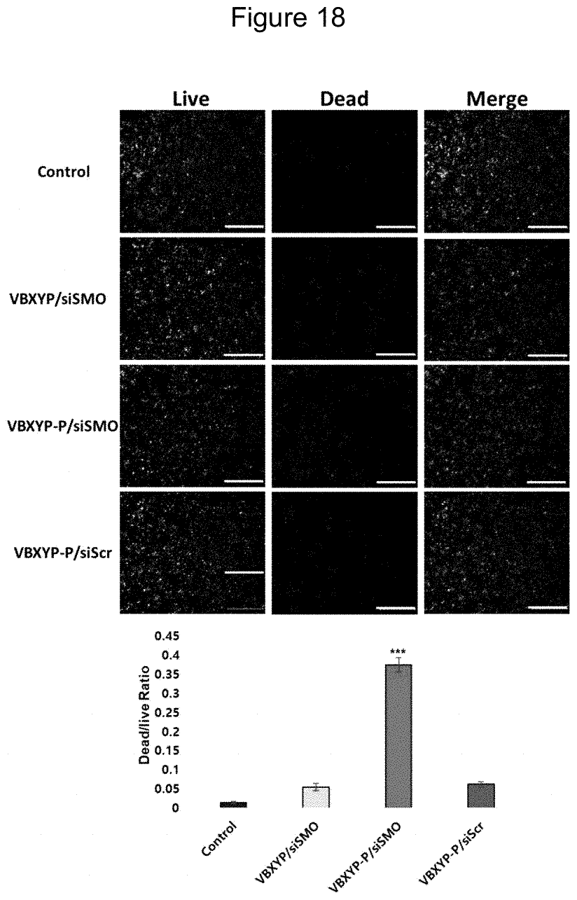

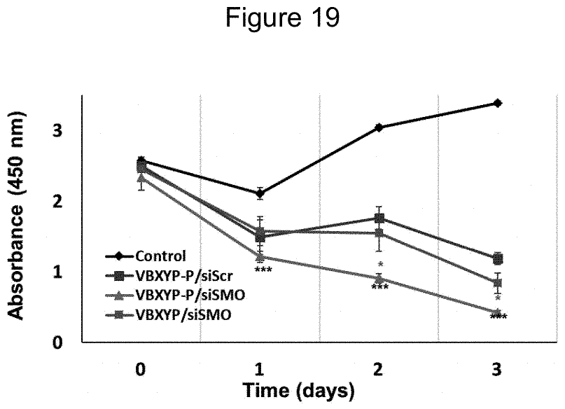

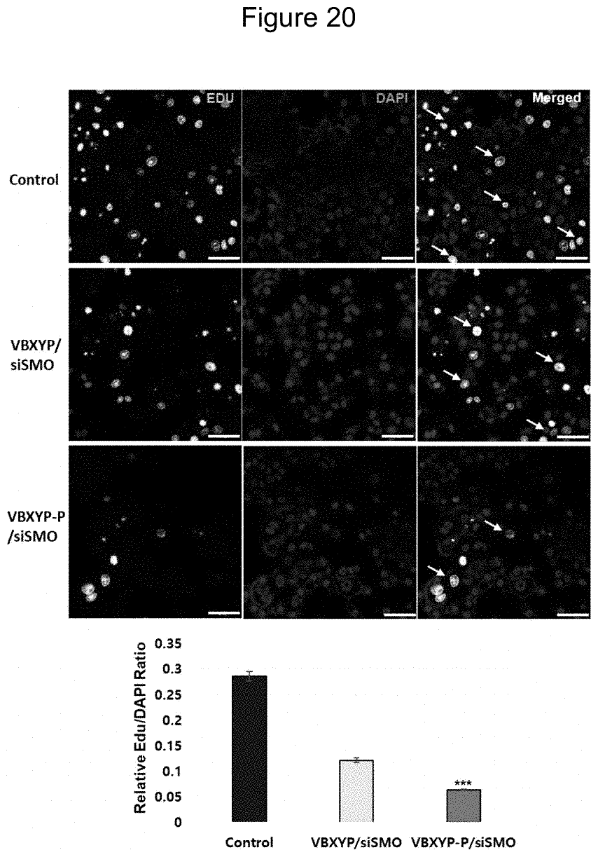

In order to confirm a change in proliferative ability of cancer stem cells after treated with VBXYP-P/siSMO, a cell live/dead assay, a WST-1 cell proliferation evaluation, and an Edu assay were performed (FIGS. 18 through 20). As a result, the same results as when VBXYP-P/SMOcr was delivered were confirmed. In the three cell proliferative ability evaluations, it was confirmed that the cell proliferation was most reduced in the experimental group of the cancer stem cells treated with VBXYP-P/siRNA. From these results, it was confirmed that known-down of SMO by siSMO delivery also induced a significant reduction in proliferative ability of the cancer stem cells, and the hypothesis that the siSMO delivery induces apoptosis of the cancer stem cells was established.

6-2. Confirmation of Induction of Apoptosis of Cancer Stem Cells by Smoothened siRNA (siSMO) Delivery

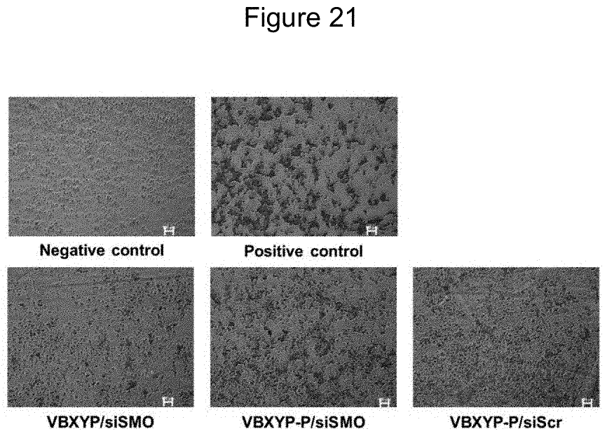

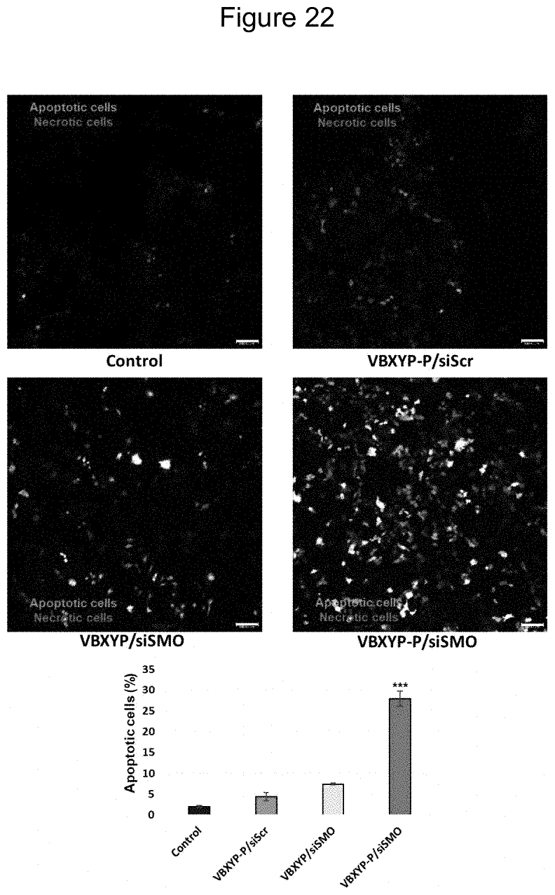

After the induction of known-down of SMO by siSMO delivery, the above-described TUNEL assay and the Annexin V assay that were used to confirm apoptosis were conducted (FIGS. 21 and 22). As a result, it was confirmed that in the experimental group in which siSMO was delivered to cancer stem cells using VBXYP-P, apoptosis occurred in the large amount of cancer stem cells.

6-3. Assay of Expression of Smoothened after Knock-Down of Smoothened

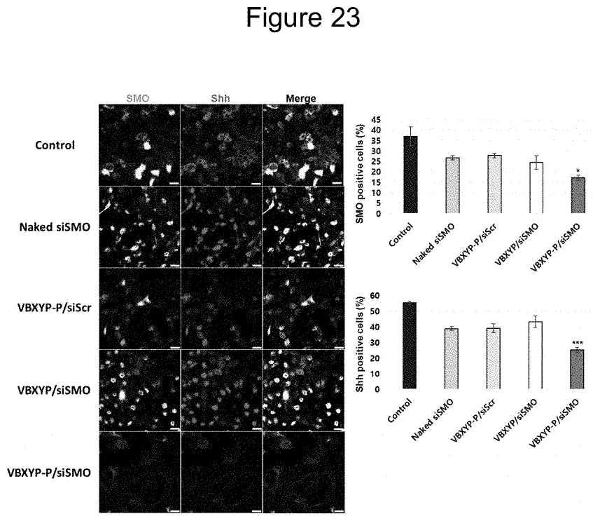

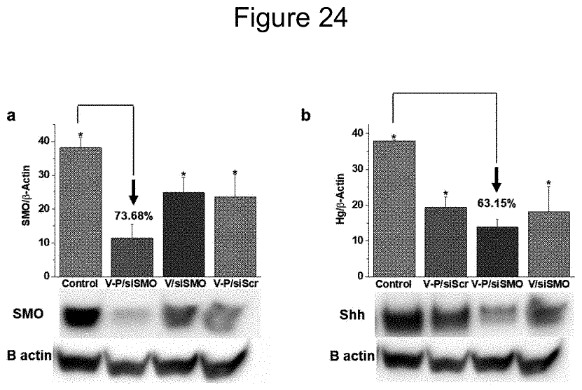

Knock-down of SMO was induced by siSMO delivery to cancer stem cells using VBXYP-P of the present disclosure, a change in expression of SMO according to the knock-down of SMO was compared and assayed using immunofluorescence and a western blot protein quantitative assay (FIGS. 23 and 24). As a result, in the experimental group of the cancer stem cells treated with VBXYP-P/siSMO, expression of each of SMO and Shh was the lowest. After comparing the experimental group treated with VBXYP-P/siSMO with the control group subjected to no treatment by the western blot protein quantitative assay, it was confirmed that the amount of SMO was reduced by about 74% and the amount of Shh was reduced by about 63%. Based on these results, the present inventors have demonstrated through the findings described above that as with the induction of apoptosis of cancer stem cells by knock-out of SMO using SMOcr, expression of Shh was reduced by knock-down of SMO in the cancer stem cells treated with VBXYP-P/siSMO, and apoptosis of the cancer stem cells were induced by disruption of the self-renewal pathways.

### Inventive Example 7: Delivery of Targeting Genes to Cancer Stem Cells in Glioblastoma Multiforme after Permeation of Blood Brain Barrier (BBB) and Brain Tumor Barrier (BTB)

7-1. Construction of BBB and BTB Simulation Models Using Three-Dimensional BBB Microfluidic Chip

Astrocytes were cultured in the central portion of a three-dimensional BBB microfluidic chip in order to simulate brain tissues, and human umbilical vein endothelial cells (HUVECs) were cultured in the outer portion of the chip in order to simulate blood vessels, and then, a medium was continuously flowed using a syringe pump to a portion corresponding to the blood vessel (0.02 to 0.5 μL/min) to induce formation of blood vessels similar to real blood vessels. In addition, glioblastoma multiforme cells or cancer stem cells were cultured in the central portion of the chip in order to simulate BTB, and umbilical vein endothelial cells were cultured in the outer portion of the chip, and then, a medium was continuously flowed to the outer blood vessel portion (0.02 to 0.5 μL/min) to induce formation of blood vessels similar to real blood vessels.

7-2. Comparison of Permeabilities of VBXYP and VBXYP-P into BBB

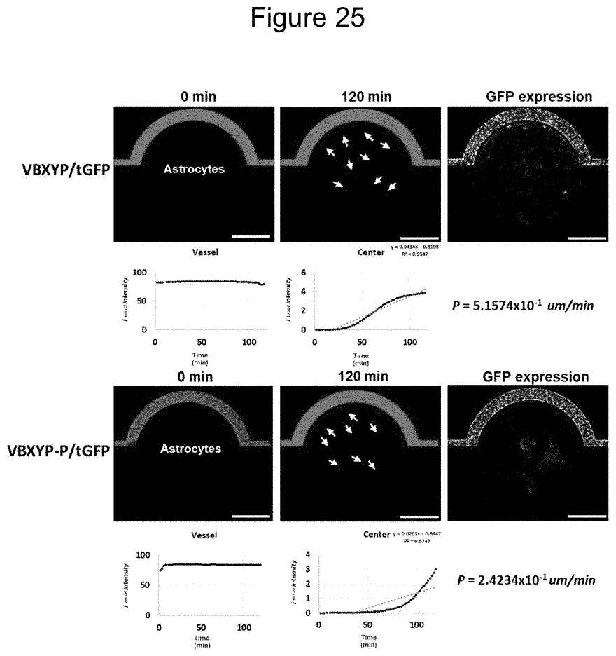

After VBXYP and VBXYP-P were labeled with TRITC to exhibit red fluorescence, a complex with GFP genes was formed, it was qualitatively confirmed how much of the complex permeated into the central portion in which astrocytes were cultured while the complex was flowed to the blood vessel portion of the three-dimensional BBB microfluidic model at a rate of 0.01 μL/min for 120 minutes, and a permeability of each of the transporters was calculated by image analysis. In addition, after 48 hours, it was confirmed that how much of transfection occurred by the corresponding gene transporter. As a result, both the VBXYP and VBXYP-P gene transporters permeated BBB, and the permeability of VBXYP into BBB was higher than that of VBXYP-P. The results of the degrees of transfection after 48 hours were similar to each other in both experimental groups (FIG. 25).

7-3. Targeting Gene Delivery of VBXYP-P to Cancer Stem Cells

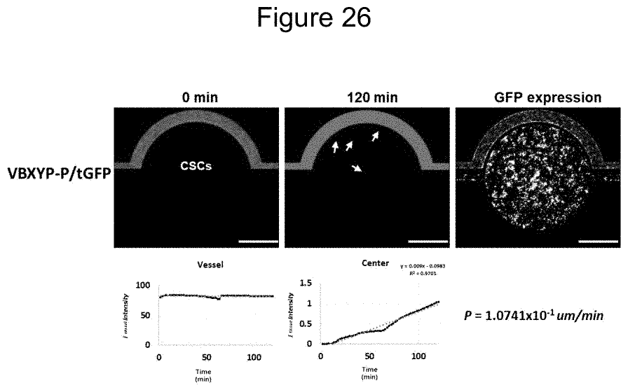

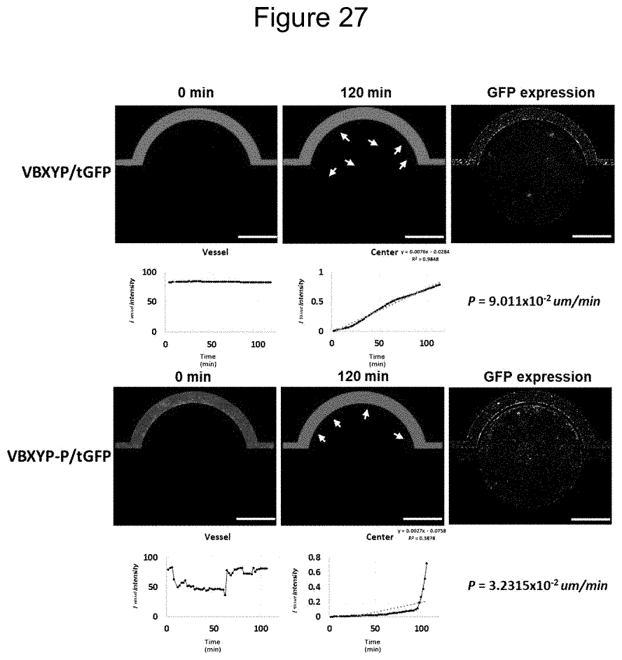

It was confirmed that how much of VBXYP-P/GFP permeated simulated blood vessels and delivered genes to cells in the central portion while VBXYP-P/GFP was flowed to the blood vessel portion in the three-dimensional BBB microfluidic chip in which cancer stem cells were cultured in the central portion at a rate of 0.01 μL/min for 120 minutes (FIG. 26). As a result, the permeability was significantly lower than when VBXYP-P was flowed to a simple BBB model. However, after 48 hours, a remarkably high degree of transfection was confirmed in the cancer stem cells compared to the transfection rate in the astrocytes. From this result, the fact that VBXYP-P has a high transfection ability for cancer stem cells, which was confirmed through the above experiment, was confirmed once again through the three-dimensional BTB model. It could be confirmed that although the permeability of the gene transporter into the tumor cells may be lowered as the tumor cells proliferate at a remarkably high density compared to the astrocytes, when the targeting functional gene transporter is used, desired genes maybe delivered to a target. Finally, glioblastoma multiforme was cultured in the central portion of the three-dimensional microfluidic chip, each of VBXYP-P/GFP and VBXYP/GFP was flowed to the blood vessel portion at a rate of 0.01 μL/min for 120 minutes, and the permeabilities of the gene transporters and the degrees of transfection after 48 hours were compared with each other (FIG. 27). As a result, it was confirmed that in comparison to when each transporter was applied to a general BBB model, the permeability was significantly lowered overall, but the permeability aspects of the respective transporters were similar to each other. Similarly, in the present model, the permeability of VBXYP/GFP was higher than that of VBXYP-P/GFP. However, after 48 hours, in the experimental group treated with VBXYP-P/GFP, remarkably highly transfected cells were found. It was demonstrated through these results of the present experiment that VBXYP-P not only could permeate BBB and BTB, but also could target cancer stem cells present inside the tumor in a significantly small amount and could deliver genes to the cancer stem cells.

The present inventors have invented the gene transporter called VBXYP-P capable of targeting cancer stem cells through all the exemplary embodiments, have demonstrated that apoptosis of the cancer stem cells could be induced using, for the transporter, smoothened (SMO) CRISPR and siRNA capable of disrupting the self-renewal signaling of the cancer stem cells and thus inducing apoptosis, and have identified the mechanism thereof. In addition, the present inventors have demonstrated through the three-dimensional microfluidic system that the gene transporter of the present disclosure not only could permeate BBB but also could target the cancer stem cells inside the brain tumor.

From the above description, those skilled in the art to which the present disclosure pertains will appreciate that the present disclosure may be embodied in other specific forms without changing the technical spirit or essential features of the present disclosure. In this regard, it is to be understood that the exemplary embodiments described hereinabove are illustrative rather than being restrictive in all aspects. It is to be understood that all modifications or variations derived from the meanings and scope of the claims described below and equivalents thereof are included in the scope of the present disclosure rather than the above-mentioned description.

### INDUSTRIAL APPLICABILITY

It is confirmed that the polydixylitol polymer gene transporter (VBXYP-P) to which vitamin B6 and a cancer stem cell-specific peptide are coupled has a remarkably higher nucleic acid delivery rate to cancer stem cells than that of the pre-existing nucleic acid transporter, has almost no cytotoxicity in a complex when bound with DNA, and above all, permeates a blood brain barrier to target cancer stem cells in glioblastoma multiforme and thus to specifically deliver the nucleic acid, thereby inducing transfection. Therefore, the gene transporter of the present disclosure inhibits expression of cancer stem cells in a tumor in vivo, and thus may be broadly used in gene therapy for various cancer diseases.

1.-19. (canceled)

20. A polydixylitol polymer gene transporter (dixylitol diacrylate VB-PEI-TR7 peptide copolymer, VBXYP-P) comprising vitamin B6 and TR-7,
wherein the polydixylitol polymer gene transporter is represented by the following Chemical Formula 1:

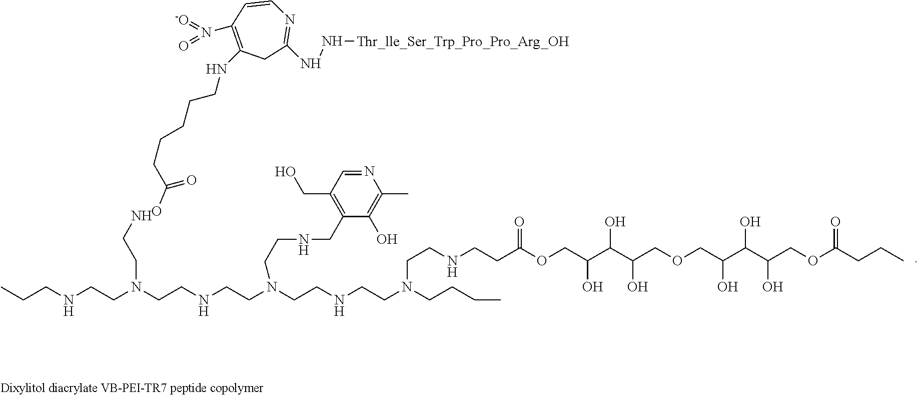

21. The polydixylitol polymer gene transporter of claim 20, wherein the transporter permeates a blood brain barrier (BBB).

22. A nucleic acid delivery complex in which a therapeutic nucleic acid is bound to the polydixylitol polymer gene transporter of claim 20.

23. The nucleic acid delivery complex of claim 22, wherein siRNA is bound as a therapeutic nucleic acid to the polydixylitol polymer gene transporter.

24. The nucleic acid delivery complex of claim 22, wherein the therapeutic nucleic acid and the polydixylitol polymer gene transporter are bound to each other at a molar ratio of 1:0.5 to 1:100.

25. The nucleic acid delivery complex of claim 22, wherein the nucleic acid delivery complex has an average particle size of 50 to 200 nm.

26. The nucleic acid delivery complex of claim 22, wherein the nucleic acid delivery complex has a zeta potential of 25 to 40 mV.

27. The nucleic acid delivery complex of claim 22, wherein the therapeutic nucleic acid is one plasmid composed of CRISPR sgRNA and Cas9 gene.

28. The nucleic acid delivery complex of claim 23, wherein the therapeutic nucleic acid is selected from the group consisting of small interfering RNA (siRNA), small hairpin RNA (shRNA), endoribonuclease-prepared siRNAs (esiRNA), antisense oligonucleotides, DNA, single-stranded RNA, double-stranded RNA, DNA-RNA hybrids, and ribozymes.

29. The nucleic acid delivery complex of claim 24, wherein the therapeutic nucleic acid is used to knock out smoothened (SMO) in cancer stem cells.

30. The nucleic acid delivery complex of claim 24, wherein the therapeutic nucleic acid is sgRNA corresponding to a sequence of “TATCGTGCCGGAAGAACTCC” identified as SEQ ID NO:2 or “AGGAGGTGCGTAACCGCATC” identified as SEQ ID NO:3.

31. The nucleic acid delivery complex of claim 23, wherein the therapeutic nucleic acid is used to knock down smoothened (SMO) in cancer stem cells.

32. The nucleic acid delivery complex of claim 23, wherein the therapeutic nucleic acid is esiRNA corresponding to Catalog No. 4392420 that is SMO siRNA (ThermoFisher) inhibiting expression of smoothened.

33. A pharmaceutical composition for gene therapy, comprising the nucleic acid delivery complex of claim 22 as an active ingredient.

34. The pharmaceutical composition of claim 33, wherein the nucleic acid delivery complex is formulated into a formulation for inhalation administration or injection administration.

35. The pharmaceutical composition of claim 33, wherein the therapeutic nucleic acid contained in the nucleic acid delivery complex targets cancer stem cells and inhibits expression of smoothened (SMO).

36. The pharmaceutical composition of claim 35, wherein the composition has an effect of treating or preventing a cancer.

37. The pharmaceutical composition of claim 36, wherein the cancer is selected from the group consisting of glioblastoma multiforme, a lung cancer, a bone cancer, a pancreatic cancer, a skin cancer, a head and neck cancer, skin melanoma, a uterine cancer, an ovarian cancer, a rectal cancer, a colorectal cancer, a colon cancer, a breast cancer, uterine sarcoma, fallopian tube carcinoma, endometrial carcinoma, cervix carcinoma, vagina carcinoma, vulva carcinoma, an esophageal cancer, a small intestine cancer, a thyroid cancer, a parathyroid cancer, soft tissue sarcoma, a urethral cancer, a penile cancer, a prostate cancer, chronic or acute leukemia, a pediatric solid tumor, differentiated lymphoma, a bladder cancer, a kidney cancer, renal cell carcinoma, renal pelvic carcinoma, primary central nervous system lymphoma, a myelencephalon tumor, brain stem glioma, and pituitary gland adenoma.


SEQUENCE LISTING (3 sequences)

SEQ ID NO: 1 - protein, 7 aa, Artificial Sequence
  TR-7
TISWPPR

SEQ ID NO: 2 - DNA, 20 bp, Artificial Sequence
  SMO CRISPR sgRNA
tatcgtgccggaagaactcc

SEQ ID NO: 3 - DNA, 20 bp, Artificial Sequence
  SMO CRISPR sgRNA
aggaggtgcgtaaccgcatc